(19) United States
(12) Patent Application Publication
(10) Pub. No.: US 2004/0235165 A1
(43) Pub. Date: Nov. 25, 2004

(54) In vitro differentiation of adult stem cells

(76) Inventors: Darwin Prockop, Philadelphia, PA (US); Jeffrey L. Spees, New Orleans, LA (US); Ichiro Sekiya, Tokyo (JP)

Correspondence Address: MORGAN, LEWIS & BOCKIUS LLP, 1701 MARKET STREET, PHILADELPHIA, PA, 19103-2921, US

(21) Appl. No.: 10/440,793

(22) Filed: May 19, 2003

(51) Int. Cl. C12N 5/00; C12N 5/08; C12N 5/02

(52) U.S. Cl. 435/377; 435/372

(57) ABSTRACT

The present invention includes a method for differentiating marrow stromal cells (MSCs) in vitro. The present invention also includes a method for improving the recovery rate of a mammal afflicted with a neuronal injury. The present invention also includes a method for enhancing the differentiation of marrow stromal cells to hypertrophic chondrocytes.

### STATEMENT REGARDING FEDERALLY SUPPORTED RESEARCH OR DEVELOPMENT

The present invention was made in part with support from grants obtained from the National Institutes of Health (Nos. AR47796 and AR44210). The federal government may have rights in the present invention.

### BACKGROUND OF THE INVENTION

Bone marrow contains at least two types of stem cells, hematopoietic stem cells and stem cells of non-hematopoietic tissues. The latter types of cells are variously referred to as mesenchymal stem cells or marrow stromal cells (MSCs). MSCs are of interest because they are easily isolated from a small aspirate of bone marrow, and they readily generate single-cell derived colonies. Single-cell derived colonies of MSCs can be expanded through as many as 50 population doublings in about 10 weeks, and they can differentiate into osteoblasts, adipocytes, chondrocytes (A. J. Friedenstein, et al. Cell Tissue Kinet. 3:393-403 (1970); H. Castro-Malaspina et al., Blood 56:289-301 (1980); N. N. Beresford, et al. J. Cell Sci. 102:341-351 (1992); D. J. Prockop, Science 276:71-74 (1997)), myocytes (S. Wakitani, et al. Muscle Nerve 18:1417-1426 (1995)), astrocytes, oligodendrocytes, and neurons (S. A. Azizi, et al. Proc. Natl. Acad. Sci. USA 95:3908-3913 (1998); G. C. Kopen, et al. Proc. Natl. Acad. Sci. USA 96:10711-10716 (1999); M. Chopp et al., Neuroreport II, 3001-3005 (2000); D. Woodbury, et al. Neuroscience Res. 61:364-370 (2000)), and cells of many other tissues (WO96/30031).

Furthermore, MSCs can give rise to cells of all three germ layers (Kopen, G. C. et al., Proc. Natl. Acad. Sci. 96:10711-10716(1999); Liechty, K. W. et al. Nature Med. 6:1282-1286 (2000); Kotton, D. N. et al. Development 128:5181-5188 (2001); Toma, C. et al. Circulation 105:93-98 (2002); Jiang, Y. et al. Nature 418:41-49 (2002). In vivo evidence indicates that unfractionated bone marrow-derived cells, as well as pure populations of MSCs can give rise to epithelial cell-types including those of the lung (Krause, et al. Cell 105:369-377 (2001); Petersen, et al. Science 284:1168-1170 (1999)) and several recent studies have shown that engraftment of MSCs is enhanced by tissue injury (Ferrari, G. et al. Science 279:1528-1530 (1998); Okamoto, R. et al. Nature Med. 8:1101-1017 (2002)). For these reasons, MSCs are currently being tested for their potential use in cell and gene therapy of a number of human diseases (Horwitz et al., Nat. Med. 5:309-313 (1999); Caplan, et al. Clin. Orthoped. 379:567-570 (2000)).

Marrow stromal cells constitute an alternative source of pluripotent stem cells. Under physiological conditions they are believed to maintain the architecture of bone marrow and regulate hematopoiesis with the help of different cell adhesion molecules and the secretion of cytokines, respectively (Clark, B. R. & Keating, A. (1995) Ann NY Acad Sci 770:70-78). MSCs grown out of bone marrow cell suspensions by their selective attachment to tissue culture plastic can be efficiently expanded (Azizi, S. A., et al. (1998) Proc Natl Acad Sci USA 95:3908-3913; Colter, D. C., et al. (2000) Proc Natl Acad Sci USA 97:3213-218) and genetically manipulated (Schwarz, E. J., et al. (1999) Hum Gene Ther 10:2539-2549).

MSCs are referred to as mesenchymal stem cells because they are capable of differentiating into multiple mesodermal tissues, including bone (Beresford, J. N., et al. (1992) J Cell Sci 102:341-351), cartilage (Lennon, D. P., et al. (1995) Exp Cell Res 219:211-222), fat (Beresford, J. N., et al. (1992) J Cell Sci 102, 341-351) and muscle (Wakitani, et al. (1995) Muscle Nerve 18:1417-1426). In addition, differentiation into neuron-like cells expressing neuronal markers has been reported (Woodbury, D., et al. (2000) J Neurosci Res 61:364-370; Sanchez-Ramos, J., et al. (2000) Exp Neurol 164:247-256; Deng, W., et al. (2001) Biochem Biophys Res Commun 282:148-152), suggesting that MSCs may be capable of overcoming germ layer commitment. Importantly, MSCs can migrate along known migration pathways when injected into the corpus striatum of rats (Azizi, S. A., et al. (1998) Proc Natl Acad Sci USA 95:3908-3913). MSCs migrated throughout forebrain and cerebellum, integrated into CNS cytoarchitecture and expressed markers typical of mature astrocytes and neurons after injection into the lateral ventricle of neonatal mice (Kopen, G. et al. (1999) Proc Natl Acad Sci USA 96:10711-10716).

### BRIEF SUMMARY OF THE INVENTION

The present invention relates to a method of in vitro differentiation of adult marrow stromal cells (MSCs). The invention also relates to uses for the differentiated MSCs in treating various diseases.

The present invention relates to a method of differentiating an adult bone marrow stromal cell into a desired cell type. The method includes co-culturing a marrow stromal cell with another cell having a desired cell type. In the method, the marrow stromal cell acquires the phenotype of said another cell.

The other cell may or may not be damaged prior to co-culturing with said adult bone marrow stromal cell. The cell damage may be caused by subjecting the other cell to a treatment selected from the group consisting of heat shock treatment, x-ray treatment, osmotic pressure, electroporation, and treatment with a toxin.

The desired cell type may be selected from the group consisting of a neuronal cell, an epithelial cell, a chondrocyte cell, a myocyte cell, an adipocyte cell, a thyroid cell, an adrenal cell, an endothelial cell, a cardiomyocyte, a renal cell, a hepatocyte, and a beta cell.

The present invention also includes the marrow stromal cell produced by practicing the method of the present invention.

The present invention also includes a method for improving recovery of a mammal from a neuronal injury. The method includes culturing bone marrow stromal cells in a neurigenic medium and implanting the cultured marrow stromal cells into the site of neuronal injury at least seven days post injury.

The neurigenic medium used in the method may include beta-3-mercaptoethanol or dimethylsulfoxide and butylated hydroxyanisole.

The present invention also includes a method of enhancing production of hypertrophic chondrocyte cells. The method includes co-culturing marrow stromal cells with chondrocytes in a hyperchondrogenic medium. The medium may include from about 10 nanomolar to about 100 nanomolar beta glycerol phosphate, or more preferably, about 20 nanomolar.

The present invention includes a hyperchondrogenic medium comprising about 20 nanomolar glycerol phosphate.

The present invention also includes a method of inducing a bone marrow stromal cell to produce a protein. The method includes transfecting the bone marrow stromal cell with a DNA construct comprising a splice site; a promoter for the expression of said protein; a marker gene; an internal ribosomal site (IRES); an antibiotic resistance gene; and another splice site; co-culturing the transfected bone marrow stromal cell with another damaged cell type obtained from an animal source; isolating those transfected bone marrow stromal cells that express both the marker gene and the antibiotic resistance gene; and optionally excising the DNA construct from said bone marrow stromal cell by incubation with recombinant Cre protein. The protein produced by the induced marrow stromal cell may be insulin when the marrow stromal cell is co-cultured with a heat-shocked beta cell.

More specifically, the present invention includes a method of inducing a bone marrow stromal cell to produce insulin. The method includes transfecting the bone marrow stromal cell with a DNA construct comprising a lox site; a promoter for the expression of insulin; a green fluorescent protein gene; an internal ribosomal site (IRES); a neomycin resistance gene; and another lox site; co-culturing the transfected bone marrow stromal cell with a heat-shocked beta cell obtained from an animal source; isolating those transfected bone marrow stromal cells that express both the green fluorescent protein gene and the neomycin resistance gene; and optionally excising the DNA construct from the bone marrow stromal cell by incubation with recombinant Cre protein.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. However, it should be understood that the invention is not limited to the precise arrangements and instrumentalities shown. In the drawings:

FIG. 1, comprising FIGS. 1A-1L, is an image of a set of phase contrast and UV microscopy photomicrographs of MSCs, small airway epithelial cells (SAECs), or MSCs co-cultured with SAECs. FIG. 1A is a SAEC culture magnified 10×. FIG. 1B is a GFP⁺ hMSCs culture grown in complete MSCs medium (FITC overlay on phase), magnification 10×. FIGS. 1C and 1D are GFP³⁰ hMSCs cultured in serum-free SAEC medium; magnification 10×. FIGS. 1E and 1F are co-cultures of MSCs with heat-shocked bronchial epithelial cells at 2 weeks. Differentiated GFP⁺ cells have epithelial morphology, have repaired the bronchial epithelium, and are bi-nucleated (arrow), as is a GFP³⁰ bronchial cell above it (arrowhead); magnification 40×. FIGS. 1G and 1H are co-cultures of GFP³⁰ hMSCs and SAECs after incubation for 12 hours. GFP³⁰ cell between SAECs undergoes morphological change (FIG. 1G arrow); magnification 20×. FIG. 1I and 1J depict a differentiated GFP³⁰ cell having epithelial morphology and a single nucleus (arrow) after 96 hours of incubation. The small airway epithelium has been repaired. The adjacent SAEC is bi-nucleated (arrowhead); magnification 20×. FIGS. 1K and 1L depict a differentiated GFP⁺ cell having three nuclei (arrow) after 120 hours incubation; magnification 40×.

FIG. 2, comprising FIGS. 2A-2U, is an image of a set of photomicrographs depicting immunocytochemistry of GFP⁺ hMSCs and SAEC co-cultures. FIGS. 2A-2O represent differentiated GFP⁺ cells expressing keratins 17, 18, 19, and CC26 (clara cells). FIGS. 2P-2U represent markers of adherens junctions, E-cadherin and beta-catenin. FIGS. 2A-2E and 2P-2Q depict UV results with a FITC filter. FIGS. 2F-2J and 2R-2S depict UV results with a TRITC filter. FIGS. 2K-2O and 2T-2U represent merged images with DAPI nuclear staining. Arrows indicate double positive cells and asterisks indicate undifferentiated GFP⁺ hMSCs. Magnification: 40×.

FIG. 3, comprising FIGS. 3A-3D represents FACS isolation of GFP⁺ cells from the co-cultures. FIG. 3A is a graph of the FACS phenotype of GFP⁺ hMSCs (gate 1) and SAECs (gate 2) from co-cultures. FIG. 3B is an image of an immunoblot for keratins 17, 18, and 19 for GFP⁺ hMSCs prior to co-culture (lane 1), SAECs (lane 2), and GFP⁺ cells isolated by FACS after co-culture with damaged SAECs (lane 3; cells isolated with gate 1 from FIG. 3A). FIGS. 3C and 3D are graphs of signal intensities of selected epithelial genes of GFP⁺ cells from co-cultures assayed by microarrays. GFP⁺ hMSCs incubated in complete MSCs medium (20% serum; hMSCs). GFP⁺ hMSCs incubated in SAEC medium (serum-free; hMSCM). GFP⁺ cells isolated from the co-cultures by FACS (EPI/DIFF) Normal airway epithelial cell gene expression (SAEC).

FIG. 4, comprising FIGS. 4A-4O, is a series of images of a set of time lapsed photomicrographs of cell fusion in GFP⁺ hMSCs and heat-shocked SAEC co-cultures. The same fields were photographed every 20 minutes by both differential interference (top panel) and UV microscopy (bottom panel). Arrows indicate GFP⁺ hMSCs. Arrowheads indicate targeted SAECs. FIGS. 4E, 4J, and 4O are enlargements of FIGS. 4D, 4I, and 4N. Arrows in FIG. 4O indicate multiple nuclei in fused cells.

FIG. 5, comprising FIGS. 5A-5C, is a graph (FIG. 5A) and an image of a set of photomicrographs. FIG. 5A is a graph depicting the sorting of GFP⁺/CD24+cells from co-cultures. FIGS. 5B and 5C represent deconvolution microscopy of GFP⁺/CD24⁺ cells nuclear-stained with DAPI. Multi-nucleated cells are indicated with arrows and cells with irregular nuclei are indicated with arrowheads. FIG. 5C is a reverse stain of FIG. 5B. Magnification: 40×.

FIG. 6, comprising FIGS. 6A-6C, is a series of images of a set of photomicrographs depicting fluorescent in situ hybridization (FISH) of GFP⁺/CD24⁺ cells isolated from co-cultures of male GFP hMSCs and female SAECs. The Y chromosome is indicated with green signal (FITC filter) and the X chromosome(s) is indicated with a red signal (TRITC filter). DNA nuclear staining is indicated by a blue signal (UV filter). FIG. 6A represents the control X/Y FISH of male normal human bronchial epithelial cells (arrow, Y chromosome; arrowhead, X chromosome). FIG. 6B represents a hybrid cell nucleus derived from fusion of 1 male GFP⁺ hMSCs nucleus with 2 female SAEC-derived nuclei (1 green signal, 5 red signals). FIG. 6C represents a hybrid cell nucleus generated from fusion of 1 male GFP⁺ hMSCs nucleus with 1 female SAEC nucleus (1 green signal, 3 red signals). It also shows a cell with one Y and one X chromosome, indicating that some cells differentiated without fusion. Inset, TRITC filter image from hybrid cell.

FIG. 7, comprising FIGS. 7A-7D, is a series of images of a set of photomicrographs depicting the appearance of MSCs in culture. FIG. 7A depicts expression of fibronectin by all MSCs during culture. Extensive deposition of fibronectin is observed in the cell cluster in the right lower corner. FIG. 7B depicts expression of vimentin. FIG. 7C depicts expression of laminin. FIG. 7D depicts expression of nestin, which is only detected in a subset of MSCs with different morphologies. In FIGS. 7C and 7D the GFP cell marker is shown together with laminin or nestin. Scale bars are 25 micrometers.

FIG. 8, comprising FIGS. 8A and 8B, depict a schematic diagram of the electrophysiological properties of a neuron-like MSCs. FIG. 8A depicts the membrane potential of a neuron-like MSCs at rest and during manually applied hyperpolarization and depolarization. FIG. 8B depicts the voltage-gated currents elicited via a voltage command stepping from −120 mV to 30 mV. Neither voltage-gated Na⁺ channels nor voltage-gated K⁺ channels are present.

FIG. 9, comprising FIGS. 9A and 9B, is a set of graphs depicting locomotor recovery as measured by BBB scores. FIG. 9A is an analysis of animals immediately treated with MSCs. Animals treated with MSCs immediately after spinal cord injury did not differ from control animals. FIG. 9B is an analysis of delayed MSCs treatment, which significantly improved locomotor recovery. (*P 0.013). Data represent mean ±S.E.M.

FIG. 10, comprising FIGS. 10A-10E, is a series of images of a set of photomicrographs depicting MSCs expression during one-week delayed transplantation of MSCs after spinal cord injury. In FIG. 10A (scale bar 250 micrometers), MSCs formed bundles bridging the epicenter of the lesion visualized by the transgenic GFP marker. Arrows indicate the location of the impact injury. In FIG. 10B, nestin immunoreactive immature astrocytes with longitudinally aligned processes were found within MSCs bundles. In FIG. 10C, GFAP marked astrocytic processes penetrating the grafted cell aggregates. In FIG. 10D, 5-HT-positive fibers were present among the implanted MSCs. In FIG. 10E, robust NF-IR nerve fiber bundles were found at the interface between MSCs and host tissue. In FIGS. 10B, 10C and 10E, asterisks indicate macrophages. In FIGS. 10B, 10C, 10D and 10E, scale bar is 25 micrometers.

FIG. 11, comprising FIGS. 11A-11F, is a series of images of a set of photomicrographs depicting immunoreactivity of MSCs-bundles. Beyond the astrocytic scar surrounding the epicenter of the lesion (FIG. 11A), FIG. 11B depicts nestin-positive and GFAP-negative immature astrocytes, which are found closely associated with transplanted MSCs, depicted in FIGS. 11C, 11D, 11E, and 11F. Neurofilament fibers are found in close relationship with nestin fibers mainly in the periphery of MSCs bundles. Scale bars are 25 micrometers.

FIG. 12, comprising FIGS. 12A, 12B, and 12C, is a set of photomicrographs depicting expression of neural markers in MSCs. FIGS. 12A and 12B depict MSCs expression of NeuN immunoreactivity at five weeks after spinal cord injury. In FIG. 12C, all MSCs were fibronectin-positive. Scale bars are 10 micrometers (FIG. 12A) and 25 micrometers (FIGS. 12B and 12C).

FIG. 13, comprising FIGS. 13A-13D, is set of images illustrating the histology of MSCs pellets stained with Safranin-O for proteoglycans at 3 weeks (FIG. 13A), 4 weeks (FIG. 13B), 5 weeks (FIG. 13C), and 6 weeks (FIG. 13D) (magnification 20×).

FIG. 14 represents an image of a photomicrograph depicting a time sequence of osteogenesis-related gene expression assayed by RT-PCR. Row 1 represents RUNX2/CBFA1; row 2 represents osterix; row 3 is integrin-binding sialoprotein (IBSP); row 4 is osteocalcin; row 5 is beta-actin.

FIG. 15, comprising FIGS. 15A-15L, is a set of graphs depicting a time sequence of osteogenesis- and chondrogenesis- related gene expressions assayed by microarray. Gene expression levels were measured as fold changes calculated from the levels in undifferentiated MSCs on day 0. FIG. 15A is COL10A1 (Genbank Accession No. X60382); FIG. 15B is osteopontin (Genbank Accession No. AF052124); FIG. 15C is cartilage oligomeric matrix protein (COMP; Genbank Accession No. L32137); FIG. 15D is aggrecan (Genbank Accession No. X17406); FIG. 15E is dermatan sulfate proteoglycan-3 (DSPG3; Genbank Accession No. U59111); FIG. 15F is Matrilin-3 (Genbank Accession No. AJ001047); FIG. 15G is prolyl 4-hydroxylase alpha-2 (P4H-alpha-2; Genbank Accession No. U90441); FIG. 15H is IBSP (Genbank Accession No. J05213); FIG. 15I is PTH/PTHrP receptor (PTHrP-R; Genbank Accession No. U17418); FIG. 15J is COL2A1 (Genbank Accession No. L10347); FIG. 15K is alkaline phosphatase (ALP; Genbank Accession No. AB011406); and FIG. 15L is osteocalcin (Genbank Accession No. AI31030).

FIG. 16, comprising FIGS. 16A, 16B, and 16C, is a set of graphs representing a time sequence of gene expression of proteinases assayed by microarray. Gene expression intensity levels were measured as fold changes calculated from the levels in undifferentiated MSCs on day 0. FIG. 16A is Cathepsin 0 (Genbank Accession No. X82153); FIG. 16B is Cathepsin H (Genbank Accession No. X16832); and FIG. 16C is MMP-8 (Genbank Accession No. J05556).

FIG. 17, comprising FIGS. 17A-17L, is a set of images of high magnification (400×) photomicrographs representing immunohistochemical analysis of pellets of MSCs cultured as micromass. FIGS. 17A and 17B represent toluidine blue in sodium borate staining for proteoglycans. FIGS. 17C and 17D represent immunostaining for type II collagen (COL2); FIGS. 17E and 17F represent immunostaining for type X collagen (COL10); FIGS. 17G and 17H represent immunostaining for IBSP; FIGS. 17I and 17J represent immunostaining for osteocalcin. FIGS. 17K and 17L represent a negative control for immunostaining that was performed without the first antibody. FIGS. 17A, 17C, 17E, 17G, 17I, and 17K were stained at 3 weeks and FIGS. 17B, 17D, 17F, 17H, 17J, and 17L were stained at 6 weeks. When samples were cultured for the same time, sequential sections were used, and about the same areas were photographed.

FIG. 18, comprising FIGS. 18A-18F, is an image of a set of lower magnification photomicrographs (100×) of immunohistochemical analysis of MSCs pellets cultured as micromass. Immunostained proteins are indicated. FIG. 18A is toluidine blue in sodium borate. FIG. 18B is a negative control (performed without the first antibody reaction). FIG. 18C is COL2; FIG. 18D is COL10; FIG. 18E is IBSP; and FIG. 18F is osteocalcin. Sequential slides were used, and the same areas were taken for photographs. The area indicated by the triangle in FIG. 18E was an artifact.

FIG. 19 represents a scheme of MSCs differentiation in micromass culture. Arrows indicate peak levels of mRNAs for transcription factors (left) and matrix-related genes (right).

FIG. 20 is a schematic diagram depicting one embodiment of the present invention, and demonstrates that MSCs can repair a damaged monolayer of epithelium either by cell fusion or differentiation without fusion.

### DETAILED DESCRIPTION

The present invention relates to methods for differentiating adult marrow stromal cells into a desired cell type to use as a therapeutic for treating a disorder. The present invention also includes a method for improving the recovery rate of a mammal afflicted with a neuronal injury. The present invention also includes a method for enhancing the differentiation of marrow stromal cells to hypertrophic chondrocytes.

Definitions

The articles “a” and “an” are used herein to refer to one or to more than one (i.e. to at least one) of the grammatical object of the article. By way of example, “an element” means one element or more than one element.

As used herein, “adult bone marrow stromal cells,” “stromal cells,” “isolated marrow stromal cells,” and “MSCs” are used interchangeably and are meant to refer to the small fraction of cells in bone marrow which can serve as stem cell-like precursors of osteocytes, chondrocytes, monocytes, and adipocytes and which are isolated from bone marrow by their ability adhere to plastic dishes. Marrow stromal cells may be derived from any animal. In some embodiments, stromal cells are derived from primates, preferably humans.

As used herein, the term “desired cell type” refers to any cell type to be co-cultured with MSCs, and to which the MSCs differentiate. For example, and not by limitation, a desired cell type may include epithelial cells, neuronal cells, chondrocytes, myocytes, adipocytes, and beta cells.

As used herein, the term “co-culture” is used to refer to at least two different types of cells being cultured together in vitro in the same culture dish. Preferably, one of the cell types is MSCs.

As used herein, the term “neuronal injury” refers to any injury or damage to a neuron or to the central nervous system. An example of a neuronal injury includes spinal cord injury.

As used herein, the term “neurigenic medium” refers to a culture medium in which marrow stromal cells are induced to express at least some markers known to be expressed by neurons and/or neuron precursors.

As used herein, the term “hypertrophic chondrocyte cell” is used to refer to an abnormally large chondrocyte cell that overexpresses chondrocytic proteins.

The term “differentiation” as used herein, should be construed to mean the induction of a differentiated phenotype in an undifferentiated cell by co-culturing the undifferentiated cell in the presence of a substantially homogeneous population of differentiated cells, in the presence of products of differentiated cells, or in the presence of an inducer of cell differentiation.

Description

The present invention includes a method of differentiating an adult marrow stromal cell to a specific desired cell type by co-culturing the MSCs with another cell having the phenotype of the desired cell type. The desired cell type may or may not be damaged. By way of example, and not by limitation, damage may be caused by heat shock treatment, x-rays, electroporation, toxins, or osmotic pressures. The desired cell type may be derived from any mammalian source. The MSCs and desired cell type are preferably co-cultured in a medium useful for culturing the desired cell type. In general, such media are well-known in the art as more fully described below. The resulting differentiated marrow stromal cells may be used to treat diseases that are a result of damage to or loss of the desired cell type. The significant advantage of this method and over the prior art methods is that it is the patient's own cells that are isolated, cultured, and returned to them after they are differentiated via co-culture with other cells. As such, rejection by the host of the newly differentiated cells (i.e., the desired cells) derived from the MSCs is not likely.

The desired cell may be useful in the patient for structural purposes, such as regenerating connective tissue. Such cells include, for example, epithelial cells and chondrocyte cells. The desired cell may also be useful to the patient for replenishing a protein that would be expressed or present at less than normal protein levels, or for providing a protein inhibitor to a patient in need of such an inhibitor. Cells which serve this purpose include, for example, beta cells, which secrete insulin. Because MSCs can differentiate into cells classed in three developmental germ layers (i.e., ectoderm, mesoderm, and endoderm), virtually any cell type may be co-cultured with the MSCs to induce differentiation of the MSCs into the desired cell type. Other types of cells that can be co-cultured with MSCs include endothelial cells, cardiomyocytes, renal cells, liver cells, neural cells, including glial cells and astrocytes, muscle cells, osteocytes, chondrocytes, adipocytes, and hepatocytes.

Treatable conditions using the technology provided herein include diabetes, thyroid conditions, such as hypothyroidism and obesity, tissue repair and regeneration, such as cartilage damage, epithelial tissue damage, nerve damage, spinal cord injury, bone injury, and brain injury, lung diseases, such as emphysema, bronchiectasis, and cystic fibrosis, vascular disease, such as arterialsclerosis and atherosclerosis, anemia, acute and chronic heart failure, liver disease, and kidney disease.

One embodiment of the present invention includes co-culturing MSCs with heat-shocked epithelial cells, such that the MSCs are induced to differentiate into epithelial cells. Preferably, the MSCs are obtained directly from the patient in which they will be used as a treatment. The MSCs expressing the epithelial cell phenotype produced by this method are useful for treating epithelial-related diseases where epithelial tissue becomes damaged. Such diseases include, but are not limited to lung diseases such as emphysema, and skin diseases.

Another embodiment of the present invention includes co-culturing MSCs from a patient with protein-secreting cells obtained from an animal source, thus inducing differentiation of the MSCs to protein-secreting cells and therefore providing the patient with their own protein-secreting cells. The resulting protein-secreting cells are returned to the patient to aid in treatment of conditions related to the deficiency in the particular protein. Such protein-secreting cells include thyroid cells, which produce thyroid hormone, pituitary cells, which produce growth hormone, adrenal cells, which produce cortisol, beta cells, which produce insulin, cells producing appetite controlling hormone, and cells secreting erythropoietin are also useful in the present invention.

One embodiment of the present invention includes co-culturing MSCs from a patient with insulin secreting beta cells obtained from an animal source, thus inducing differentiation of the MSCs to insulin-secreting cells and therefore providing the patient with their own insulin secreting cells. The resulting insulin-secreting cells are returned to the patient to aid in treatment of diabetes and diabetes-related conditions.

In one embodiment of the present invention, differentiated MSCs may be produced by transfecting the MSCs with a DNA construct comprising: a splice site, such as the Lox site; a promoter for the expression of a specific protein (which protein is expressed by a cell of interest); a marker gene, such as green fluorescent protein (GFP) gene; an internal ribosomal site (IRES); an antibiotic resistance gene, such as neomycin resistance; and another splice site. Transfected MSCs are co-cultured with a heat-shocked cell of interest obtained from an animal source, such as a pig or rat. The MSCs expressing the marker gene along with the antibiotic resistance gene are isolated. Optionally the DNA construct is excised from the cells by incubating the cells with recombinant Cre protein. The excision of the DNA construct may be important in treating human patients because recent evidence indicates that cells expressing the neomycin resistance gene are rapidly destroyed in humans. Thus, excision of the DNA construct negates the possibility that transformed MSCs may be destroyed before rendering any benefit to the human patient.

Since DNA construct preparation and transfection are so well known in the art, it would be within the skilled artisan's purview to determine an appropriate splice site, marker, resistance gene, and promoter to use in preparing a construct for use with the present invention.

In one embodiment of the present invention, MSCs are transfected with a construct that comprises a Lox site, a promoter for insulin, a GFP gene, an IRES, a neomycin resistance gene, and another Lox site, in a 5′ to 3′ orientation. The MSCs may be co-cultured for a period of time with heat-shocked (or otherwise damaged) pig pancreatic islet cells (beta cells), during which time the MSCs differentiate into pancreatic islet cells. The MSCs expressing GFP and demonstrating neomycin resistance are isolated, for example, by treating the cell culture with neomycin. Optionally, the MSCs are subjected to incubation with Cre protein to excise the DNA construct (including the neomycin resistance gene). The cells are then administered to the patient in need.

In another embodiment of the present invention, MSCs are co-cultured with and/or fused with beta cells to produce human somatostatin and/or a human glucose receptor.

One of skill in the art would know which culture medium is preferred for successful co-culture of MSCs with the particular cell type of interest. For example, a neuronal induction medium, such as the medium discussed in more detail elsewhere herein, is suitable for co-culturing MSCs and neurons. In any event, MSCs media containing serum should be used to culture MSCs.

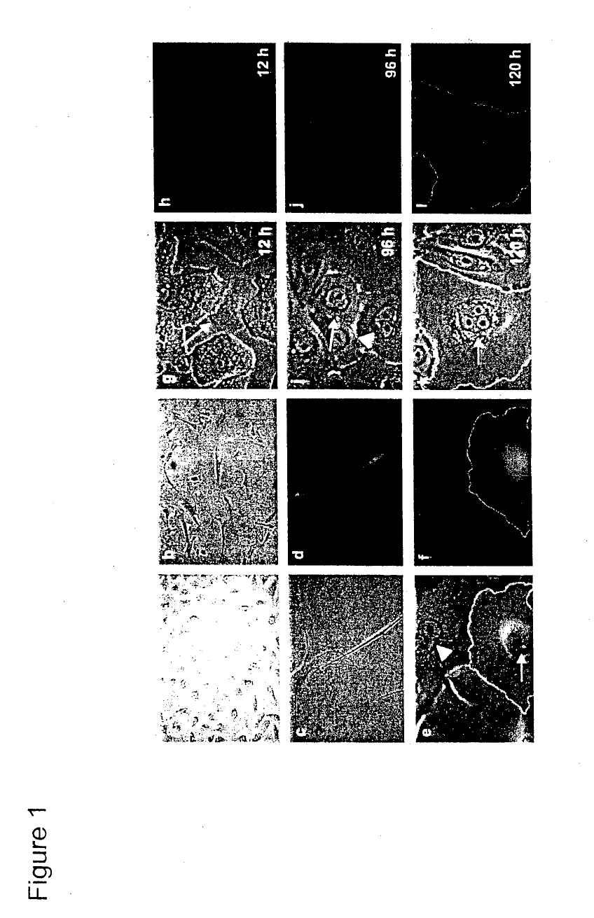

The cell culture time and conditions are also available in the art to the skilled person. Certain cell types must be co-cultured with MSCs for longer periods of time than other cell types to achieve differentiation of the MSCs into the cell type of interest. For example, MSCs co-cultured with epithelial cells differentiate rapidly. MSCs begin to lose their fibroblast morphology about 12 hours after culture, and become flattened and translucent and adopt an epithelial shape (see FIGS. 1G and 1H). After 24 hours, many of the MSCs are indistinguishable from the epithelial cells when viewed under phase contrast microscopy. By 48 to 96 hours, the epithelial cell culture to which MSCs were added re-assembles to a continuous monolayer.

Differentiated MSCs can be administered to a patient in need in a variety of ways. For example, MSCs can be delivered directly to the site of tissue damage via injection or implantation. MSCs can also be delivered systemically and/or parenterally.

The present invention also includes a method for improving recovery in a mammal receiving MSCs to treat a neural defect. The method includes culturing the marrow stromal cell in an appropriate neurigenic medium and implanting the MSCs into a neuronal defect site, for example, a spinal cord injury site, at least one week after the injury occurs. An example of a neurigenic medium includes Dulbecco's modified eagle medium (DMEM) (Sigma-Aldrich; St. Louis, Mo.) supplemented with 2% dimethylsulfoxide (DMSO) and 200 micromolar butylated hydroxyanisole (BHA). Another example is DMEM with 5 millimolar beta-3-mercaptoethanol (beta-3-ME). Other examples of neurigenic media include those disclosed in Woodbury, et al., J. Neurosci. Res. 61:364-370 (2000). Although MSCs cultured in either of these example media do not express all of the genes that normal neurons express, the MSCs demonstrate an ability to improve recovery from a neuronal injury.

This delayed treatment is shown in the present invention to greatly improve recovery following neural injury. For example, in Example 2, paraplegic rats having a spinal cord injury were treated with MSCs by implantation of the MSCs at the injury site either immediately or seven days post-injury. The MSCs delivered seven days post injury improved recovery of the rats from the spinal cord injury. The implanted MSCs were able to form bundles and guide regenerating neuropil through the spinal cord lesion.

In another embodiment of the present invention, a method for differentiating MSCs into hypertrophic chondrocytes is taught. The method comprises culturing MSCs in a chondrogenic medium for a period of time, followed by culturing the MSCs in a hyperchondrogenic medium for another period of time. The chondrogenic medium preferably comprises TGF-beta-3, BMP-6, and dexamethasone. Preferably, the MSCs are cultured for about 3 weeks, but may be cultured from about 21 to about 120 days, before being transferred to the hyperchondrogenic medium.

The MSCs are also cultured in hyperchondrogenic medium. The medium preferably includes about 10 to about 100 nanomolar beta-glycerol phosphate, and more preferably, includes about 20 nanomolar beta-glycerol phosphate. This amount of beta-glycerol phosphate is about one million-fold less than the typical amount present in a hyperchondrogenic medium. Surprisingly, this one million-fold decrease in beta-glycerol phosphate concentration greatly enhances the number of hypertrophic chondrocytes produced compared with the conventional method for producing hypertrophic chondrocytes.

In addition to presenting a hypertrophic chondrocyte phenotype, the MSCs may also acquire an osteoblast phenotype as discussed more fully below in Example 3. Immunohistochemistry data indicate that MSCs cultured in this way produce cells that express both chondrocyte and osteoblast markers.

The following examples are provided to illustrate the present invention. It should be understood, however, that the invention is not to be limited to the specific conditions or details described in these examples. Throughout the specification, any and all references to a publicly available document, including but not limited to a U.S. patent, are specifically incorporated by reference.

### EXAMPLES

### Example 1

### Differentiation of Marrow Stromal Cells into Epithelial-like Cells

To investigate human adult stem cell differentiation in response to tissue injury, an ex vivo model of human adult stem cells from bone marrow stroma (hMSCs) co-cultured with heat-shocked human small airway epithelial cells (SAECs) was developed.

Briefly, the results demonstrate that a subset of the hMSCs rapidly differentiated into epithelial-like cells, and they restored the epithelial monolayer of damaged epithelial tissue. Immunocytochemistry and microarray analyses established that the hMSCs co-cultured with SAECs expressed many genes characteristic of normal small airway epithelial cells. Some hMSCs differentiated directly after incorporation into the epithelial monolayer but other hMSCs fused with epithelial cells.

In addition, because two recent reports suggested that cell fusion may explain some of the observed plasticity of adult stem cells, the co-cultures were examined for evidence of cell fusion (Terada, et al, Nature, 416:542-545 (2002); Ying, et al. Nature, 416:545-548 (2002)). Surprisingly, cell fusion was a frequent rather than rare event, ranging from 1% as assayed by a conservative flow-cytometric assay to 14% as assayed by time-lapse microscopy. Nuclear fusion also occurred in some cells.

The materials and methods used in the experiments presented in this Examples are now described.

Cell Culture and Manipulation

hMSCs were obtained from 2 to 10 milliliters of iliac crest bone marrow aspirates (right and left side) from a normal male human donor. Clinical procedures were performed after informed consent and approval by an institutional review board. Isolation and culture conditions were as described previously in Sekiya, et al., Proc. Natl. Acad. Sci. 99:4397-4402 (2002). Adherent hMSCs were propagated in complete medium: alpha-MEM, 2 millimolar L-glutamine, 100 units per milliliter penicillin, 100 micrograms per milliliter streptomycin (GIBCO, Rockville, Md.), supplemented with 20% FCS (lot selected for rapid growth of hMSCs; Atlanta Biological, Norcross, Ga.). Passage 1 (P1) hMSCs were electroporated with pIRESneo (Clontech, Palo Alto, Calif.) modified to express enhanced Green Fluorescent Protein (GFP) from the cytomegalovirus (CMV) promoter. Transfected cells were selected by growth in complete medium with the addition of 200 micrograms per milliliter G418. A single clone with stable GFP expression was isolated, expanded (P2), and 3×10⁵ cells were used to seed a 6,000 cm² Cell Factory (Nunc, Rochester, N.Y.). Cultured GFP⁺ hMSCs were detached with 0.25% trypsin/1 millimolar EDTA, resuspended in phosphate buffered saline (PBS), and phenotyped by fluorescence-activated cell sorting (FACSVantage SE, Becton Dickinson, Lincoln Park, N.J.). About 95% of the cells expressed GFP. All GFP⁺ hMSCs used in this study were from the original clone and passage 4 (P4).

Primary cultures of SAEC (Clonetics, Baltimore, Md.) were grown to confluence in T-75 flasks at 37° C. and 5% CO₂ in small airway cell basal medium (SABM™, Clonetics, Baltimore, Md.) supplemented with 5×10⁻⁴ micrograms per milliliter human recombinant epidermal growth factor, 5×10⁻⁴ milligrams per milliliter epinephrine, 1×10⁻² milligrams per milliliter transferrin, 5×10⁻³ milligrams per milliliter insulin, 1×10⁻⁴ micrograms per milliliter retinoic acid, 6.5×10⁻³ micrograms per milliliter triiodothyronine, 5×10² milligrams per milliliter gentamycin, and 5×10² micrograms per milliliter amphotericin-B. Culture medium was changed every other day for optimal growth.

SAEC cultures were heat-shocked by partial emersion in a water bath (Isotemp215. Fisher Scientific, Malvern, Pa.) equilibrated to 47° C. for 30 minutes. These flasks were washed with 70% ethanol and returned to the incubator. GFP⁺ hMSCs were lifted with trypsin/EDTA at 37° C. for 5 minutes. Trypsin was deactivated by addition of complete medium and cells were centrifuged at 450 ×g for 10 minutes and resuspended in SAEC medium. One hour after heat-shock, from 2.5 to 5.0×10⁵ GFP⁺ hMSCs were added to the SAEC cultures and the cells were co-cultured at 37° C. with 5% CO₂ for up to 4 weeks. SAEC medium was changed every 2 to 3 days. Similar conditions were used with bronchial epithelial cells incubated in bronchial epithelial cell medium (BEGM™, Clonetics, Baltimore, Md.).

Microscopy and Immunocytochemistry

For immunocytochemistry, SAEC medium was aspirated and the co-cultures were washed with 1×PBS and fixed with 4% paraformaldehyde at 4° C. for 5 minutes. After three PBS washes, microscope slide-sized pieces were cut from T-75 culture flasks with a hot scalpel. Immediately after excision, the flask slides were placed into 1×PBS to hydrate them and they were subsequently stored in the dark at 4° C. An IMMEDGE™ pen (Vector Laboratories, Burlingame, Calif.) was used to form waterproof edges on the plastic slides. Slides were blocked in 5% (v/v) normal goat serum (Sigma, St. Louis, Mo.) and 0.4% (v/v) Triton X-100 in 1×PBS for 1 hour at room temperature. Individual slides were incubated in the following antibodies (Chemicon, Temecula, Calif.) overnight at 4° C.: mouse anti-keratin 17 (MAB1677, 1:200); mouse anti-keratin 18 (MAB 1600, 1:400); mouse anti-keratin 19 (MAB1607, 1:100); rabbit anti-clara cell protein 26 (AB3700, 1:400); mouse anti-beta catenin (MAB2081, 1:400); and mouse anti-E-cadherin (MAB3199, 1:200). Isotype controls were mouse IgG1 (CBL600, Cymbus Biotechnology, Chandler's Ford, UK) and mouse IgG (purified whole molecule, PP54, Chemicon, Temecula, Calif.), blocked and incubated as above. After three 5 minute PBS washes, all slides were incubated with goat anti-mouse or goat anti-rabbit ALEXA 594 (1:800, Molecular Probes, Eugene, Oreg.) for 1 hour at room temperature. Following three 5 minute PBS washes, the slides were air-dried, cover-slipped (DNA Vectashield, Vector Laboratories, Burlingame, Calif.), and photographed (Nikon Eclipse E800, SPOT RT CCD, Nikon, Japan). GFP⁺/CD24⁺ cells isolated by FACS were examined by deconvolution microscopy with a Leica DMRXA microscope equipped with an automated x, y, z stage and CCD camera (Sensicam, Intelligent Imaging Innovations, Denver, Colo.). Co-localization of multiple nuclei in single cells was confirmed by analysis of deconvoluted images taken at 1.0 micrometer intervals (Slidebook software, Intelligent Imaging Innovations, Denver, Colo.).

Immunoblotting

Following 3 weeks of co-culture, GFP⁺ hMSCs and SAEC were collected as above and flow-sorted. GFP⁺ cells were gated conservatively. GFP⁺ . hMSCs and SAEC were cultured separately for controls. Following flow-sorting, 50,000 cells were pelleted by centrifugation at 2,600 ×g for 10 minutes and stored at -70° C. Cell pellets were resuspended in 13 microliters of PBS to which 5 microliters of NuPage LDS sample buffer (4×) and 3 microliters of 2-mercaptoethanol (Sigma, St. Louis, Mo.) were added. The resuspended pellets were heated to 100° C. for 2 minutes and separated by electrophoresis on 4% to 12% NuPage bis-Tris gels with MES buffering (25,000 cell lysates per lane).

Following electrophoresis, the gels were electroblotted to PVDF membranes. All electrophoresis and electroblotting used Novex reagents and systems (Invitrogen Corporation, Carlsbad, Calif.). The blots were blocked overnight at 4° C. in 5% non-fat dry milk in PBST (PBS with 0.1% (v/v) Tween 20), washed 3 times for 5 minutes each in PBST, and incubated in 1:1,000 primary antisera (Chemicon, Temecula, Calif.) in PBST for 1 hour at room temperature. After three 5 minute washes in PBST, the blots were incubated in 1:2,000 goat-anti-mouse IgG-Horseradish Peroxidase (HRP) conjugate (Sigma, St. Louis, Mo.) in PBST for 1 hour at room temperature. Unbound secondary antibody was removed and positive bands were detected with a chemiluminescent reaction.

Microarray

hMSCs were co-cultured with damaged SAEC for 2 weeks as above. GFP⁺ cells were flow-sorted into PBS. Total RNA was isolated with an RNeasy kit (Qiagen, Valencia, Calif.). Experimental procedures for GeneChip microarray were performed according to the Affymetrix GeneChip Expression Analysis Technical Manual (Affymetrix, Santa Clara, Calif.). In brief, 8 micrograms of total RNA was used to synthesize double-stranded DNA (Superscript Choice System, GIBCO/BRL Life Technologies, Rockville, Md.). The DNA was purified by using phenol/chloroform extraction with Phase Lock Gel (Eppendorf® Scientific, Westbury, N.Y.) and concentrated by ethanol precipitation. In vitro transcription was performed to produce biotin-labeled cRNA by using a BioArray HighYield RNA Transcription Labeling Kit (Enzo Diagnostics, Farmingdale, N.Y.). Biotinylated cRNA was cleaned with an RNeasy Mini Kit (Qiagen, Valencia, Calif.), fragmented to 50 to 200 nucleotides, and hybridized for 16 hours at 45° C. to Affymetrix HG-U95Av2 array, which contains approximately 12,500 human genes. After washing, the array was stained with streptavidin-phycoerythrin (Molecular Probes, Eugene, Oreg.). Staining signal was amplified by biotinylated anti-streptavidin (Vector Laboratories, Burlingame, Calif.) and by a second staining with streptavidin-phycoerythrin. The chip was then scanned on a Hewlett-Packard GeneArray Scanner. The expression data were analyzed using Affymetrix MicroArray Suite v5.0. Signal intensities of all probe sets were scaled to the target value of 2500.

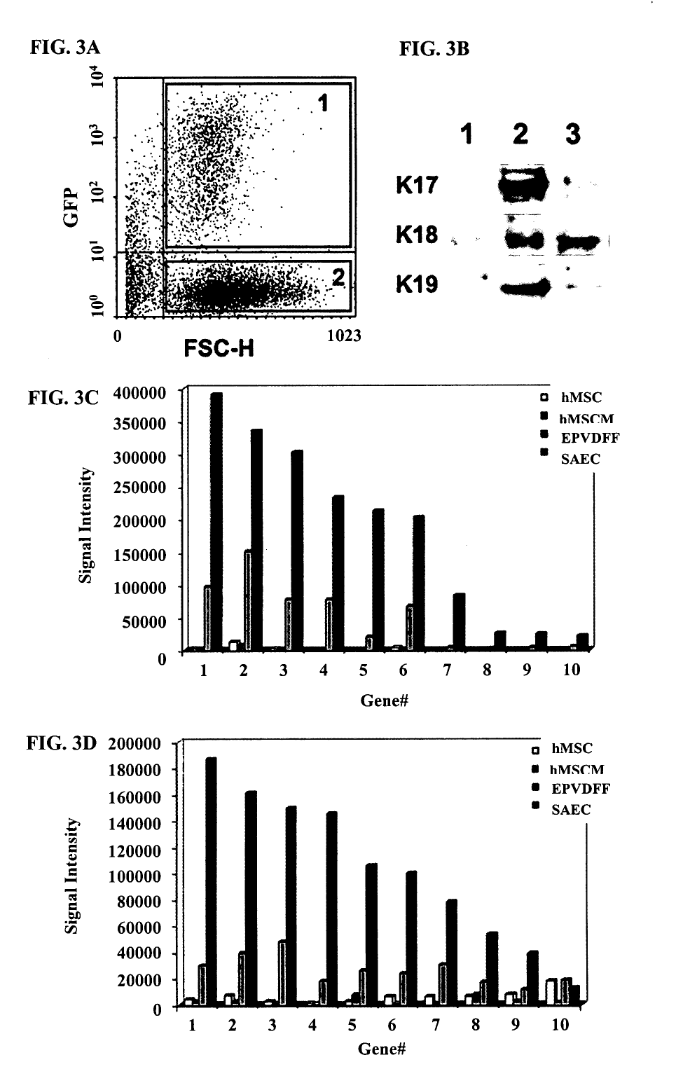

Statistical analyses were performed with SPSS software (version 11.0 windows) and Fisher's z-transformation and one sample z-test. Accession numbers for genes displayed in FIG. 3: (1) Stratifin (GenBank Accession No. X57348), (2) Keratin 17 (GenBank Accession No. Z19574), (3), Keratin 6 (GenBank Accession No. L42611), (4) Kenatin type II (GenBank Accession No. M21389), (5) Keratin 19 (GenBank Accession No. Y00503), (6) CAN19 (GenBank Accession No. M87068), (7) Keratin 16 (GenBank Accession No. 28439), (8) Maspin (GenBank Accession No. U043 13), (9) CD24 (GenBank Accession No. L33930), (10) Claudin-7 (GenBank Accession No. AJ011497), (11) Cornified envelope precursor (GenBank Accession No. AF001691), (12) Laminin S B3 chain (GenBank Accession No. U17760), (13) Integrin beta 4 (GenBank Accession No. X53587), (14) E-cadherin (GenBank Accession No. Z35402), (15) Laminin-related protein (GenBank Accession No. L34155), (16) Lung amelioride sensitive Na-channel protein (GenBank Accession No. X76180), (17) P-cadherin (GenBank Accession No. X63629), (18) Laminin gamma-2 chain precursor (GenBank Accession No. Z15008), (19) NES-1 (GenBank Accession No. AF055481), (20) Mucin 1 (GenBank Accession No. X80761).

Time Lapse Microscopy

Images were obtained using an inverted microscope (Eclipse TE 200; Nikon) and CCD camera (ORCA ER; Hamamatsu). Microscope functions and filterwheels (LEP) were controlled with software (MetaMorph; Universal Imaging Technology, Downingtown, Pa.). The microscope system was enclosed in a plexiglass environmental chamber to maintain temperature at 37° by circulation of heated air. A humidified atmosphere of 5% CO₂, and 2% O₂ was maintained in an on-stage glass chamber. Images were processed by pseudo-coloring and overlaid into 24-bit RGB images. A confluent monolayer of SAECs in chamber slides (Lab-Tek™ CC2; Nunc, Rochester, N.Y.) and GFP⁺ hMSCs were cultured as described. SAECs were scanned prior to addition of GFP⁺ hMSCs and subsequently at 20 minute intervals for 4 days. The recorded images were analyzed by three independent observers.

Fluorescent in Situ Hybridization

GFP⁺/CD24⁺ cells were isolated from co-cultures female GFP⁺ hMSCs and female SAECs by FACS as above. Cells were sorted into chamber slides with SAEC medium and allowed to adhere overnight. The next day, they were washed 3 times with PBS and fixed for 5 minutes in ice cold 100% methanol. X and Y chromosome FISH was then performed with the manufacturer's protocol (CEP X SpectrumOrange™/Y SpectrumGreen™ DNA Probe Kit ;Vysis, Downers Grove, Ill.). The CEP X DNA probe (DXZ1 locus) hybridizes to alpha satellite DNA at the centromeric region of the X chromosome (Xp11.1-q11.1). The CEP Y DNA probe (DZY1 locus) hybridizes to satellite III DNA at the Yq12 region of the Y chromosome.

TABLE 1
Nuclear characteristics of isolated GFP+/CD24+ cells

| | Total cell | Bi- | Tri- | Single | Irregular |
|---|---|---|---|---|---|
| Experiment | number | nucleated | nucleated | nuclei | nuclei |
| 1 | 366 | 96(26%) | 12(3.3%) | 136(37%) | 122(33%) |
| 2 | 246 | 56(23%) | 5(2.0%) | 93(38%) | 92(37%) |
| 3 | 152 | 40(26%) | 5(3.3%) | 24(16%) | 83(55%) |

Epithelial Morphology of Differentiated hMSCs

Primary human SAECs grown in defined serum-free SAEC medium formed an integrated confluent monolayer of large, flat cells with an elevated perinuclear region (FIG. 1A). Green fluorescent protein-expressing hMSCs (GFP⁺ hMSCs) were grown in complete culture medium containing 20% fetal calf serum (FCS), and displayed a fibroblast-like phenotype (FIG. 1B). When grown alone in SAEC medium, GFP⁺ hMSCs replicated slowly and developed long, thin processes after a few days (FIGS. 1C and 1D).

To test the hypothesis that hMSCs might respond to tissue injury, confluent cultures of SAECs were heat-shocked at 47° C. for 30 minutes to induce cell damage and death. Following heat shock, the majority of SAECs remained adherent but many cells lost cell-cell contact as their cytoplasms retracted, opening up holes in the monolayer.

GFP⁺ hMSCs were added 1 to 2 hours after the heat-shocked SAEC cultures had cooled to 37° C. Within 12 hours, about 1% of the adherent hMSCs began to lose their characteristic fibroblast morphology, and became flattened and translucent with an epithelial shape (FIGS. 1G and 1H). After 24 hours, many of the GFP⁺ hMSCs were indistinguishable from SAECs by phase contrast microscopy. By 48 to 96 hours, the cultures to which hMSCs were added re-assembled to a continuous monolayer (FIGS. 1I to 1L). In contrast, heat-shocked SAECs cultured alone did not consistently regain confluency. Also, GFP⁺ hMSCs added to cultures of SAECs that were not heat-shocked adhered to the surface of the monolayers, primarily at junctions between adjacent SAECs, and showed little evidence of differentiation after several days. Similar morphologic changes were observed when GFP⁺ hMSCs were used to prepare co-cultures with heat-shocked bronchial epithelial cells (FIGS. 1E and 1F).

In addition, the appearance of multi-nucleated GFP⁺ cells (see arrows in FIGS. 1E and 1K) was noted, raising the possibility of cell fusion. Many unmodified SAECs and bronchial epithelial cells were also multi-nucleated (see arrowheads in FIG. 1E and 1I).

Immunocytochemistry for Differentiation Markers

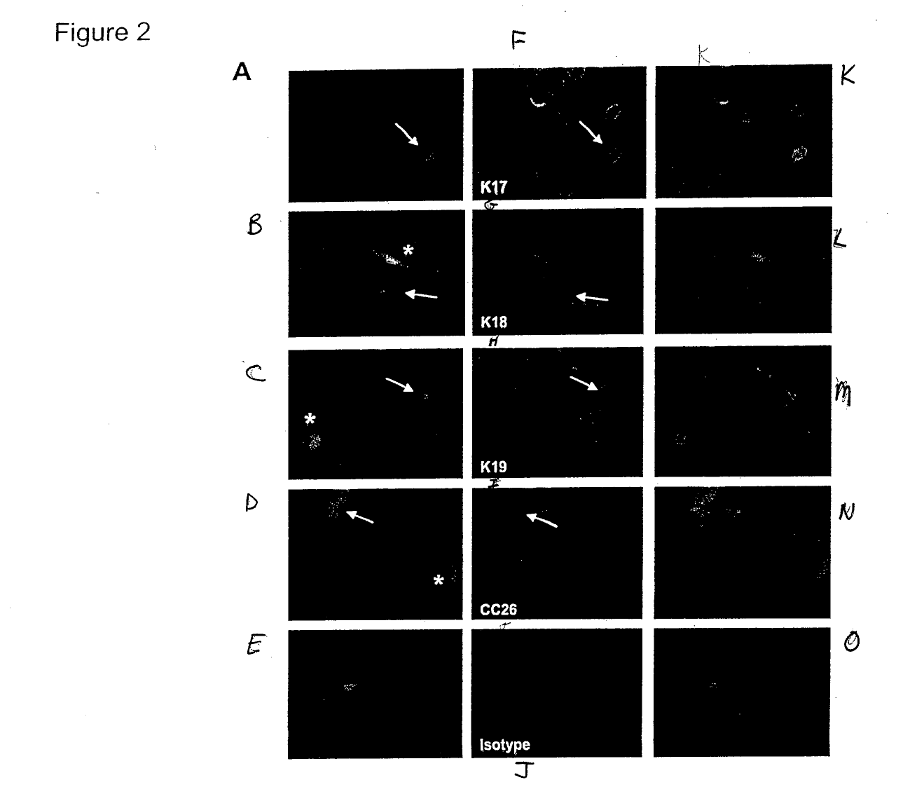

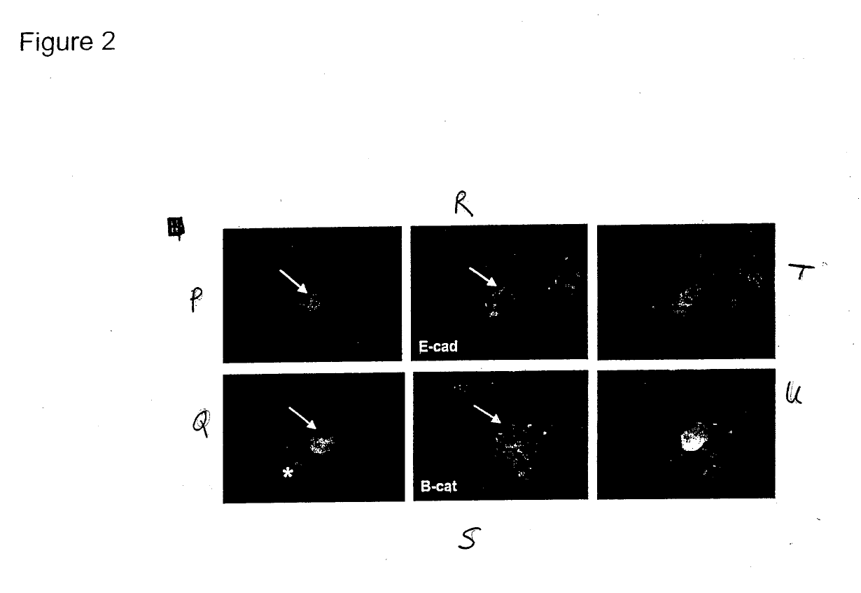

The morphologically differentiated GFP⁺ hMSCs were positive for several epithelial-specific markers including keratins 17, 18, and 19, as well as CC26, a marker present on clara cells, serous cells, and goblet cells in the lung (FIG. 2A). Additionally, immunocytochemistry for E-cadherin and beta-catenin demonstrated that differentiated GFP⁺ hMSCs formed adherens junctions with SAECs (FIG. 2B). Many cells that stained for differentiation markers were multi-nucleated.

The undifferentiated GFP⁺ hMSCs in the same co-culture were negative for keratins and CC26 (see asterisks in FIG. 2A). Also, the undifferentiated GFP⁺ hMSCs did not stain for E-cadherin, but did stain very lightly for beta-catenin. They also did not form the pseudostratified epithelioid associations characteristic of SAECs (see asterisk in FIG. 2B). The undifferentiated GFP⁺ hMSCs contained single nuclei.

Phenotypes of GFP⁺ Cells Isolated from Co-cultures

To follow differentiation, the co-cultures were sorted by FACS to isolate both differentiated and undifferentiated GFP⁺ cells from the cultures (FIG. 3A). By Western blot assays, the isolated GFP⁺ cells from 3 week old co-cultures expressed keratins 17, 18, and 19 (lane 3 in FIG. 3B); whereas GFP⁺ hMSCs cultured in complete medium expressed only low levels of keratin 18 (lane 1 in FIG. 3B). SAECs expressed all three keratins (lane 2 in FIG. 3B).

Microarray Analysis

To determine the extent of differentiation, mRNA microarrays were used to assay the total population of both differentiated and undifferentiated GFP⁺ cells from the co-culture. For analysis of the data, the genes with the highest signal intensities were scanned first, and 20 genes were selected that are characteristically expressed by epithelial cells (FIG. 3C). Although 15% or less of the cells had differentiated morphologically, correlation analysis of the 20 selected genes indicated a highly significant relationship in expression between the total GFP⁺ population (EPI/DIFF) and the SAECs (Spearman rank correlation, two-tailed test at alpha=0.01, r=0.8617, p=0.000001). Differentiated GFP⁺ cells (in EPI/DIFF) express many of the genes expressed by normal SAECs: (1) Stratifin, (2) Keratin 17, (3) Keratin 6, (4) Kenatin type II, (5) Keratin 19, (6) CAN 19, (7) Keratin 16, (8) Maspin, (9) CD24, (10) Claudin-7, (11) Cornified envelope precursor, (12) Laminin S B3 chain, (13) Integrin beta 4, (14) E-cadherin, (15) Laminin-related protein, (16) Lung amelioride sensitive Na-channel protein, (17) P-cadherin, (18) Laminin gamma-2 chain precursor, (19) NES-1, (20) Mucin 1. Expression of these genes is absent on low in GFP⁺ hMSCs cultured in complete MSCs medium (hMSCs) or in GFP⁺ hMSCs cultured in SAEC medium (hMSCM).

Next a one sample z-test (two-tailed, alpha=0.05) was performed for all possible two-way comparisons of r values (six r values, 15 comparisons). Five of the six r values were found to be statistically indistinguishable. The remaining r for the correlation of gene expression of GFP⁺ cells isolated from co-cultures (EPI/DIFF) with that of SAECs was found to be statistically greater than each of the other five. The results of these analyses support the hypothesis that the gene expression profile of GFP⁺ cells isolated from co-cultures with SAECs more closely resembles that of SAECs than any of the control samples.

Interestingly, microarray analyses of undifferentiated hMSCs revealed the expression of several transcripts commonly found in epithelial cells such as keratin 8 (signal intensity 16,466; GenBank Accession No. X14487), cytokeratin 10 (signal intensity 10,491; GenBank Accession No. X74929), and keratin 18 (signal intensity 50,650; GenBank Accession No. M26326). The mRNAs of these genes are not normally present in differentiated mesenchymal cells and are suggestive of the ability of hMSCs to differentiate across cell lineage boundaries. The microarray data corroborate those of a previous study in which a single cell-derived colony of undifferentiated hMSCs was analyzed by microSAGE and found to express keratins 8 and 10, transcripts from endothelial and epithelial cells (Tremain, et al., Stem Cells, 19:408-418 (2001)).

Time Lapse Microscopy

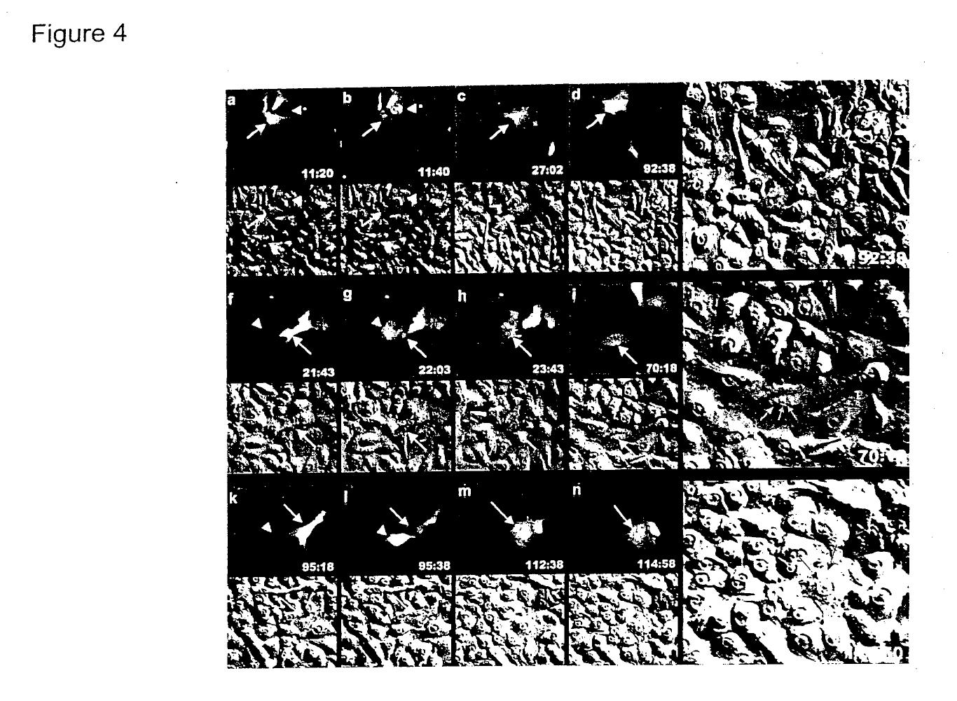

In further experiments, GFP⁺ hMSCs were added to heat-shocked SAECs and the co-cultures were photographed at 20 minute intervals for four consecutive days (FIG. 4). hMSCs were observed to adhere within 1 hour of plating. Within 24 hours, GFP⁺ hMSCs with single nuclei were observed to approach and contact SAECs (target cells). Some cells differentiated directly after incorporation into the epithelial monolayer but other GFP⁺ hMSCs fused with epithelial cells. In some instances, the GFP⁺ hMSCs were observed to extend a process to the target cells just prior to the fusion event. During cell fusion, targeted cells rapidly became GFP⁺ within 20 minute intervals (FIGS. 4B, 4G, 4L). The hybrid cells were motile and typically seen as a single large flat cell with two nuclei (arrows in FIG. 4E). Over several hours, they re-organized so that both nuclei were adjacent to one another in the elevated perinuclear region characteristic of epithelial cells. Several cells were observed to have three or more nuclei (arrows in FIG. 4J). The GFP intensity in many of the hybrid cells was typically reduced relative to undifferentiated GFP⁺ hMSCs, but GFP continued to be expressed for up to 4 weeks. Therefore, there was continuing expression of genes from the nucleus derived from the GFP⁺ hMSCs. Three hundred eighty one GFP⁺ cells were reverse tracked in the co-cultures after 4 days. At least 53 (14%) had participated in cell fusion with an SAEC.

Assays of Differentiated GFP⁺/CD24⁺ Cells Isolated from Co-Cultures

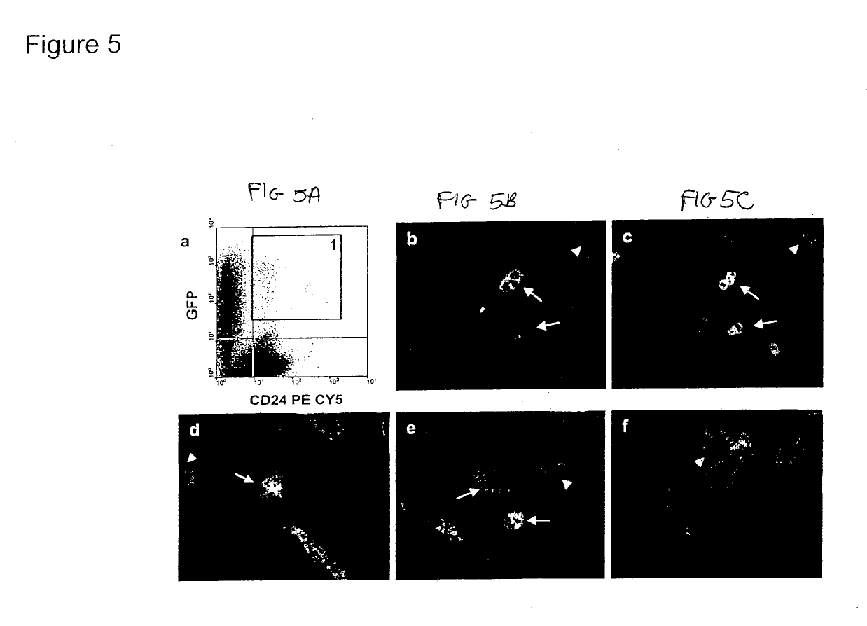

To isolate differentiated GFP⁺ cells from the co-cultures, an antibody to CD24, a mucin-like glycoprotein that is a marker for epithelial cells and not expressed on hMSCs was employed (FIG. 5A). After 48 hours, 1.3% of the co-cultured GFP⁺ cells were GFP⁺/CD24⁺ After 1 week, 4% of the co-cultured GFP⁺ cells were GFP⁺/CD24⁺. For further examination, GFP⁺/CD24⁺ cells from 1 week co-cultures were sorted into chamber slides, fixed, and nuclear-stained with DAPI (FIGS. 5B to 5F, Table 1). Of a total of 754 cells examined from three experiments, 23% to 26% were bi-nucleated, 2.0% to 3.3% were tri-nucleated, 16% to 38% had a single nucleus, and 33% to 55% had a large on irregular nucleus, suggesting nuclear fusion (Table 1).

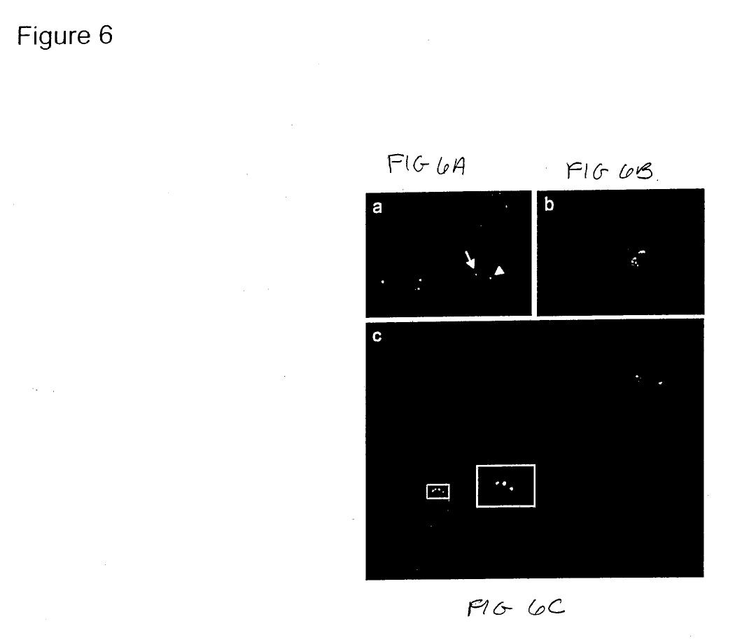

The isolated GFP⁺/CD24⁺ cells were also examined by fluorescent in situ hybridization (FISH) for the X and Y chromosomes (FIG. 6). Nuclei of GFP⁺/CD24⁺ cells isolated from co-cultures of male GFP⁺ hMSCs and female SAECs showed evidence of both nuclear fusion and single-cell differentiation. Nuclei were observed with 1Y chromosome and 5 X chromosomes, indicating that 1 male stem cell nucleus had fused with 2 female SAEC-derived nuclei (FIG. 6B). Other nuclei from the same FACS isolation possessed 1 Y chromosome and 3 ×chromosomes, indicating that 1 male stem cell nucleus had fused with 1 female SAEC-derived nucleus (FIG. 6C). In addition, nuclei from isolated GFP⁺/CD24⁺ cells were observed with 1 Y chromosome and 1 X chromosome, indicating that single-cell differentiation also occurred.

### Example 2

### Differentiation of Marrow Stromal Cells into Neuronal Cells

Primary Marrow Stromal Cell Cultures

MSCs were collected from the femurs and tibias of adult male Lewis rats (Harlan, Indianapolis, Ind.). Rats were euthanized with a mixture of 70% CO₂ and 30% O₂. Tibias and femurs were placed on ice in minimal essential medium with alpha modification (α-MEM, Gibco-BRL, Rockville, Md.) containing 20% fetal calf serum (FCS, Atlanta Biologicals), 2 millimolar L-glutamine (Gibco-BRL, Rockville, Md.), 100 units per milliliter penicillin, 100 micrograms per milliliter streptomycin, and 25 nanograms per milliliter amphotericin B (penicillin, streptomycin and amphotericin, Gibco-BRL, Rockville, Md.). Epiphyses of femur and tibia were removed and the marrow was flushed out using a syringe filled with medium. Bone marrow was filtered through a 70 micron nylon mesh and plated in 75-cm² flasks. About 24 hours after plating, supernatant containing non-adherent cells was removed and fresh medium was added. After the cells had grown to near confluency, they were passaged two to five times by being detached (0.25% trypsin/1 millimolar EDTA for 5 minutes) and replated at a density of approximately 5000 cells/cm².

Preparation of the Retroviral Vector, Production of Viral Particles and Genetic Marking of MSCs

A retroviral construct encoding green fluorescent protein (GFP) as an expression marker and aminoglycoside phosphotransferase as a neomycin (G 418) selectable marker was prepared using the LXSN vector (Clontech, Palo Alto, Calif.). Phoenix amphotropic packaging cells (ATCC) were transfected with the LXSN-GFP plasmid using calcium phosphate precipitation. Viral supernatants were collected 48 hours after the start of the transfection, filtered through a 0.45 micron filter, and stored at −80 ° C. for further use. Phoenix packaging cells were analyzed at the time of viral harvest for GFP expression.

One day before the infection of MSCs with GFP-retrovirus, about 100,000 MSCs were plated in 21.0 cm² plates. At the time of infection, defined as Day 1, 2.5 milliliters of complete medium containing 20% heat-inactivated fetal calf serum (FCS) was added to the cells in the presence of 500 microliters viral supernatant and 8 micrograms polybrene per milliliter (Sigma, St. Louis, Mo.). On Day 2 the infection procedure was repeated. On Day 3, fresh complete medium was added with 20% FCS (not heat-inactivated). On Day 4, cells were split 1:3 in 55.0 cm² plates in complete medium containing 200 micrograms G418 per millilter (Sigma, St. Louis, Mo.) for a selection period of 14 to 21 days. MSCs that had stably integrated the transgene survived and were expanded for experiments by passaging cells 3 to 9 times.

Attempts at Differentiation of MSCs Toward a Neuronal Fate MSCs were plated at a density of 2500 cells/cm². On the following day the medium was replaced with pre-induction medium consisting of Dulbecco's modified eagle medium (DMEM) (Sigma-Aldrich), 20% FCS, and 10 nanograms per milliliter bFGF. After 24 hours the pre-induction medium was removed, the cells were washed twice with PBS, and neuronal induction medium containing DMEM supplemented with 2% dimethylsulfoxide (DMSO) and 200 micromolar butylated hydroxyanisole (BHA) was added. Alternatively, DMEM with 5 millimolar beta-3-mercaptoethanol (beta-3-ME) can be used as the neuronal induction medium for the same incubation times.

Electrophysiological Recordings of Neuron-like MSCs

Whole-cell recordings were made of MSCs exhibiting possible neuronal morphologies such as rounded cell bodies and distinct processes with growth cone-like terminal expansions. Such differentiated cells will be referred to herein as neuron-like MSCs. Whole-cell recordings were obtained by using a patch-clamp amplifier (Axopatch 200 A, Axon Instruments, Union City, Calif.). The recordings had a series resistance ranging from 4 to 10 MOhms that was compensated for electronically by 75-85%. The resting membrane potential was assessed in current clamp mode. The residual capacity was removed, but the linear leak was not subtracted. To investigate the existence of voltage-gated channels, neuron-like MSCs were clamped at −120mV and currents were evoked by 100 ms depolarizing voltage steps to +30mV. The cells were perfused through a gravity-driven microperfusion system with the nozzle positioned close to the recorded cell.

The control solution was used at room temperature and contained 140 millimolar NaCl, 4 millimolar KCl, 1.8 millimolar CaCl₂, 1 millimolar MgCl₂, 23 millimolar sucrose, 10 millimolar HEPES. The pH was adjusted to 7.40 and the osmolarity to 310 mOsm. Recordings were made with pipettes of 3 to 7 MOhms filled with a solution containing 4 millimolar NaCl, 140 millimolar KCl, 0.5 millimolar CaCl₂, 1 millimolar MgCl₂, 10 millimolar HEPES and 5 millimolar EGTA. The pH was adjusted to 7.40 and the osmolarity to 305 mOsm. Membrane currents and voltages were controlled with appropriate software (PCLAMP, Axon Instruments, Union City, Calif.). Current and voltage signals were sampled at 10 kHz.

MSCs transplantation into the injured spinal cord

A total of 38 adult female Lewis rats (Charles River, Wilmington, Mass.) weighing 250-260 grams received a standardized contusion of the spinal cord and MSCs treatment immediately or one week after injury. Laminectomy was performed at T9 vertebrae under halothane anesthesia (Fluothane®, AstraZeneca, Waltham, Mass.). The impact rod of the NYU device was centered above T9 and dropped from a height of 25 millimeters.

MSCs grown under normal culture conditions were detached and resuspended with alpha-MEM to a final concentration of 30,000 viable cells per microliter as determined by trypan blue dye exclusion. Immediately or 7 days after injury, animals received 5 microliters of a MSCs suspension or alpha-MEM delivered into the injury center, and two 2.5 microliter deposits, one 2 millimeters cranial and the other 2 millimeters caudal of the central injection. A total of 300,000 cells or vehicle was thus delivered at a rate of 0.5 microliters per minute by means of a stereotaxic frame and a glass pipette with a tip diameter of 100 microns configured to a 10 microliter Hamilton syringe. Muscle and skin were sutured separately. Urinary bladders were manually emptied 5 times per day for the first week and twice daily thereafter. Antibiotics (Borgal®,Hoechst, Kansas City, Mo.) were given to prevent urinary tract infection. Two independent experiments with time-matched controls were carried out. A total of 16 rats received MSCs (N=8, N=number of animals) or cell culture medium (N=8) immediately after injury. A second group (N=22) was treated with MSCs (N=12) or vehicle (N=10) one week after injury. In both groups behavior was assessed on a weekly basis, and histological examinations were carried out on animals euthanized 5 weeks after injury. All experiments had been approved by the Animal Research Committee of Stockholm.

Behavioral Testing

Hindlimb motor function was assessed using the open field BBB scoring system. Individual rats were placed on an open field (75×125 cm), and observed for 4 minutes by two observers. Hindlimb function was scored from 0 to 21 (flaccid paralysis to normal gait). The test was carried out one day postoperatively and once every week up to the fifth week after spinal cord injury (SCI).

Cell Quantification

GFP-positive cell profiles containing a distinct nucleus were counted in serial sections. Cell numbers were calculated according to the formula of Abercrombie (Anat. Rec. 94:239-247 (1946)).

Immunocytochemistry

Tissues and cells were processed for indirect immunocytochemistry. Animals were deeply anesthetized with Pentobarbital and intracardially perfused with 50 milliliters Tyrode's solution containing 0.1 milliliter of Heparin, followed by 200 milliliters fixative (4% parafomaldehyde and 0.4% picric acid in PBS). Spinal cords were dissected, postfixed in similar fixative for one hour, transferred to 10% sucrose solution, frozen and cut in a cryostat at 14 micron thickness. Longitudinal sections were collected from 18 millimeters long spinal cord segments containing the injury and injection sites and thaw-mounted on gelatin-coated slides. MSCs were grown in chamber slides (Nunc Lab-Tek™, Rochester, N.Y.) and fixed with 4% paraformaldehyde for 10 minutes. Antisera raised in goats against fibronectin (Calbiochem®, La Jolla, Calif.) and GFP (Rockland, Gilbertsville, Pa.) or in rabbits against nestin (kindly provided by Dr. Urban Lendahl from the Karolinska Institute in Stockholm), laminin (Sigma, St. Louis, Mo.), GFAP (Sigma), neurofilament (NF), PGP 9.5 (Biogenesis, Bournemouth, UK), GFP (Molecular Probes, Eugene, Oreg.) and 5-HT (Sigma, St. Louis, Mo.) as well as mouse monoclonal antibodies to vimentin (DAKO, Carpinteria, Calif.), NeuN (Chemicon, Temecula, Calif.), NF200 (Sigma, St. Louis, Mo.) and Map-2 (Sigma, St. Louis, Mo.) were used. Secondary antisera were conjugated with FITC, rhodamine or Cy5 (Jackson Immunoresearch, West Grove, Pa.). Optimal dilutions were established for all primary and secondary antibodies. Controls included omitting the primary antibody. Slides were evaluated using epifluorescence and confocal microscopy (Radiance 2100, Bio-Rad, Hercules, Calif.)

Statistical Analysis

Comparisons of cell survival were made using an unpaired T-test. Between group comparisons for behavior were carried out using the Mann Whitney U-test. Significance levels were designated *p<0.05, **p<0.01, ***p<0.001. All values are given as mean +/− SEM.

The Results of the experiments presented in the Example are now described.

Labeling and Characterization of MSCs in vitro

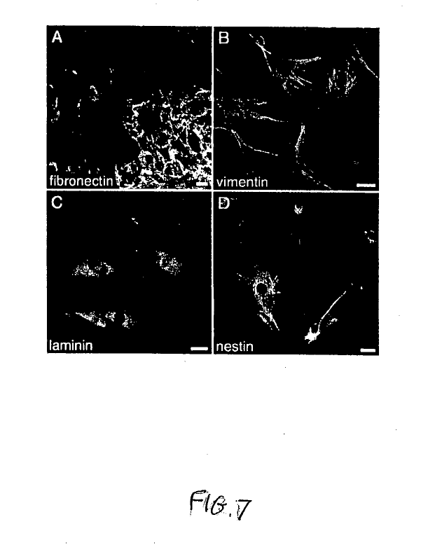

Reliable detection by fluorescence microscopy of marrow stromal cells in culture and after transplantation was achieved by transducing MSCs with a retrovirus encoding green fluorescence protein (GFP). After a selection time of 14 days in media supplemented with a cell-toxic concentration of G418, only cells that had permanently integrated plasmids containing a neomycin selectable marker survived. The successful labeling of all surviving cells was confirmed by fluorescence microscopy. Genetically labeled MSCs did not alter their morphology compared with native MSCs. All analyzed cells (n>500) were positive for fibronectin, vimentin and laminin (FIG. 7A-C, Table 1). In areas of high cell density fibronectin immunoreactive filaments were extensively deposited in the extracellular space (FIG. 7A). Immunreactivity (IR) for the mesodermal intermediate filament vimentin was dense in cellular processes, and present in the form of a filamentous meshwork in cell bodies (FIG. 7B). A distinct subpopulation of MSCs (37.5% ±1.2, n=2777) were nestin-IR (FIG. 7D). MSCs were negative for the neuron-specific markers NeuN, NF, Map-2, and PGP 9.5.

TABLE 1
IR-markers of MSCs in vitro and following spinal cord implantation.

| Marker | MSCs in vitro | Implanted MSCs |
|---|---|---|
| fibronectin | +++ | ++ |
| vimentin | ++ | − |
| laminin | + | − |
| nestin | ++ | − |
| | (37.5% ± 1.2) | |
| NeuN | − | + |
| NF | − | − |
| GFAP | − | − |
| Cell shape | flat | spindle-shaped |

− no signal, +++ strong signal

Electrophysiological properties of neuron-like MSCs

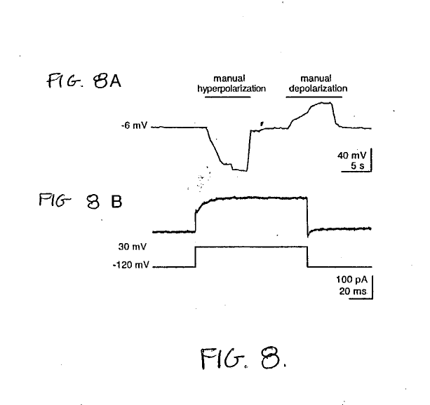

In one experiment, MSCs were induced with medium containing 2% DMSO and 200 micromolar BHA for 48 hours. However, patch clamp recordings of these neuron-like MSCs were not possible. This could have been due to changes in the cell membrane caused by dramatic changes in the osmolarity from 646 mOsm to 310 mOsm when replacing the differentiation media by the extracellular solution. In a second experiment, MSCs were differentiated with medium containing 5 millimolar beta-3-ME for 48 hours. This treatment was compatible with whole-cell recordings. The resting membrane potential was −11.4±8.7 mV (n=8). It was not possible to induce action potentials in any cell by application of depolarizing current (FIG. 8A). Using the voltage step protocol to activate voltage-gated currents, no inward currents could be elicited, indicating that the MSCs did not express functional sodium channels. An outward current amplitude was found to be 203±194.5 pA (n=8) (FIG. 8B). The low amplitude of the outward current and the presence of unavoidable leaks associated with the recording and leak currents make it very unlikely that the observed current represents a voltage-gated outward potassium current. Hence MSCs differentiated by beta-3-ME did not show typical neuronal properties such as action potentials or voltage-gated Na⁺ and F currents and are therefore not mature neurons.

Delayed Implantation of MSCs into the Injured Spinal Cord Improves Functional Recovery

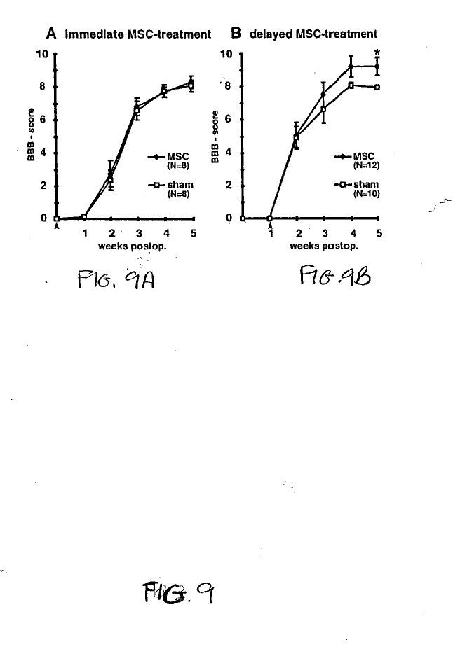

Immediate MSCs treatment did not improve locomotor function, as revealed by BBB scoring (FIG. 9A; Barso, et al., J. Neurotrauma 12:1-21 (1995)). Delayed implantation led to significantly improved BBB scores (9.2±0.5) compared to sham grafted animals (7.9±0.1) (MannWhitney U-test; p=0.013) (FIG. 9B). Five weeks after injury, control animals could not support their body weight with their hindlimbs (N=10), whereas seven animals of the treatment group (N=12) could lift their trunks and two of them regained stepping patterns with bilateral weight support and frequent forelimb-hindlimb coordination assessed as 13 on the BBB scale.

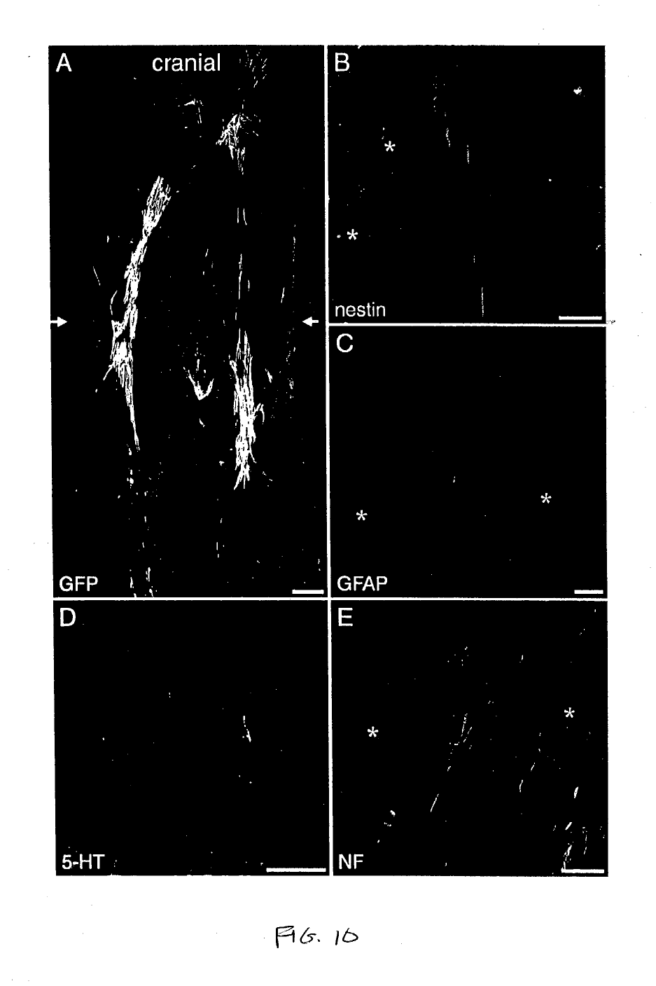

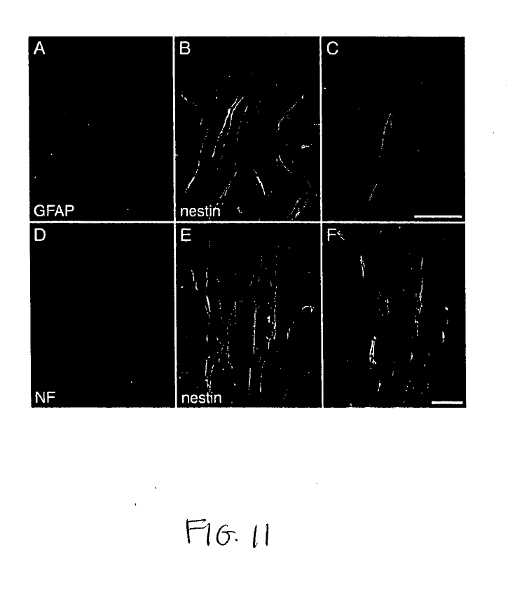

Cryostat sections were examined five weeks after SCI. MSCs were reliably detected by their GFP labeling, which was abundant in the whole cell body. Cell counts revealed significantly larger numbers of cells (2966±681, N=8) in animals treated one week after injury than in animals treated immediately (518±106, N=8) (unpaired T-test; P=0.0052). MSCs infused immediately after SCI were mainly found in the periphery of the injury zone, whereas MSCs transplanted one week after SCI were found in the whole lesion zone (FIG. 10A).

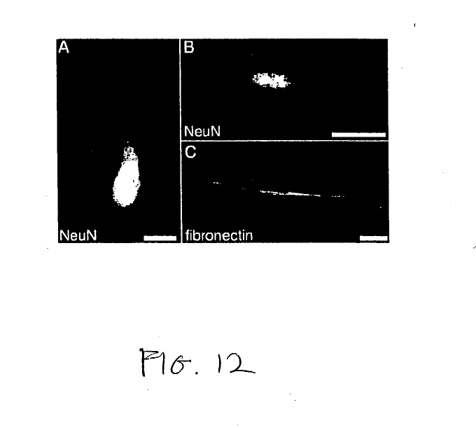

Implanted MSCs exhibited a bipolar morphology with long processes extending along the axis of the spinal cord. MSCs formed bundles, which were mainly arranged along the long axis of the spinal cord and provided bridges across the epicenter of the lesion area which was filled with debris and macrophages. All implanted MSCs expressed fibronectin-IR and a weak but distinct NeuN-IR (FIG. 12A-12C, Table 1). Interestingly, implanted MSCs had lost detectable nestin-IR (FIG. 10B, 11), as well as vimentin and laminin-IR (Table 1).

Nestin and GFAP antibodies revealed the presence of two different kinds of glial cells in the injured spinal cord. GFAP and nestin-positive reactive astrocytes delineated the margin of the epicenter of the lesion with their tightly interwoven processes. In animals that had received a MSCs infusion, astrocytic processes reached into the epicenter by penetrating MSCs-bundles (FIG. 10C).

A second population of cells was nestin-positive but GFAP-negative and thus similar to immature astrocytes. These cells had migrated into the epicenter of the injury. In animals treated with MSCs, immature astrocytes populated the MSCs bundles and extended their delicate processes along the engrafted cells (FIG. 10B, 11A-11C). NF-positive fibers were preferentially found at the interface between MSCs bundles and scar tissue (FIG. 10E, 11D-11F). Some of the nerve fibers associated with the implanted cells were identified as 5-HT-positive (FIG. 10D). The intraspinal MSCs did not display GFAP, NF, MAP-2 or PGP 9.5 immunoreactivity.

Also, 5-HT-positive nerve fibers were identified along the MSCs bundles. The 5-HT-system of the spinal cord has been shown to be important in functional recovery after SCI, and the apparent regeneration of 5-HT elicited by MSCs may thus be contributing to the observed improvement of behavioral recovery. From a clinical standpoint it is perhaps particularly encouraging that delayed MSCs treatment enhanced survival of grafted cells and exerted a beneficial effect on functional recovery. MSCs infused immediately after SCI encounter a hostile environment characterized by ischemia, necrosis and the presence of potentially toxic compounds such as oxygen radicals and lytic enzymes. However, 12 hours after SCI maximal tissue loss is reached leading to the next phase characterized by reactive gliosis, invasion of inflammatory cells and reparative attempts, such as upregulation of bFGF.

Five weeks after transplantation into an injured spinal cord, the MSCs exhibited down-regulation of vimentin, laminin and nestin and began to express a weak nuclear NeuN immunoreactivity, indicating that they were instructed by environmental cues present in the injured spinal cord to differentiate into neurons. Importantly, transplanted MSCs formed bundles bridging the epicenter of the lesion filled with debris and macrophages. Regenerating host neuropil was associated with MSCs aggregates and thus a degree of cellular organization had been reestablished in the injury zone. Immature astrocytes, defined as nestin-positive and GFAP-negative cells, which are formed from stem cells in response to injury, populated the MSCs-bundles. These cells might help promote nerve fiber outgrowth by offering a growth permissive surface. Growth of nerve fibers on the surface of astrocytes has been observed in other studies where peripheral nerves or fibroblasts secreting NGF were implanted. Another explanation for the guidance of nerve fibers might be the abundant expression of N-cadherin which is known to enhance neurite extension, on the surface of MSCs.

The data indicate that this later phase spinal cord injury provides a more habitable environment for infused MSCs. Autologous treatment might thus become possible, avoiding graft rejection, the risk of viral antigens and possible ethical concerns associated with other sources of stem cells. In sum, the results demonstrate that MSCs survive well in the contused and severely pathological tissue present in the lesion after spinal cord injury, and form physical nerve fiber-permissive tissue bridges across areas of debris, associated with a degree of long-term functional improvement.

### Example 3

### Marrow Stromal Cell Differentiation into Hypertrophic Chondrocytes

Isolation and cultures of human MSCs

To isolate human MSCs, bone marrow aspirates of 10 millilters were taken from the iliac crest of normal adult donors after informed consent and under a protocol approved by an Institutional Review Board. Nucleated cells were isolated with a density gradient (Ficoll-Paque, Pharmacia, Piscataway, N.J.) and resuspended in complete culture medium (alpha-MEM (GIBCO BRL, Rockville, Md.); 20% fetal bovine serum (FBS) lot-selected for rapid growth of MSCs (Atlanta Biologicals, Norcross, Ga.); 100 units per milliliter penicillin; 100 micrograms per milliliter streptomycin; and 2 millimolar L-glutamine (GIBCO BRL, Rockville, Md.). All of the nucleated cells (30 million) were plated in 25 millilter medium in a culture dish and incubated at 37° C. with 5% CO₂. After 24 hours, non-adherent cells were discarded, and adherent cells were thoroughly washed twice with phosphate-buffered saline (PBS). The cells were incubated for 8 days in fresh medium, harvested with 0.25% trypsin/1 millimolar EDTA for 5 minutes at 37° C., and replated at 6 cells/cm² in an intercommunicating system of culture flasks (6320 cm² Cell Factory, Nunc, Rochester, N.Y.). After 12 days, the cells (passage 1) were harvested with trypsin/EDTA, suspended at 1×10⁶ cells per milliliter in 5% DMSO and 30% FBS, and frozen in 1 milliliter aliquots in liquid nitrogen. To expand a culture, a frozen vial of MSCs was thawed, plated in a 60 cm² culture dish, and incubated for 4 days (passage 2). The cells were harvested and diluted for further expansion by plating at initial densities of 50 cells/cm² in a 180 cm² culture dish. The cells were then harvested after 7 days (passage 3).

Micromass culture of MSCs

Approximately 200,000 MSCs (passage 3) were placed in a 15 milliliter polypropylene tube (Falcon), and centrifuged at 450 ×g for 10 minutes. The pellet was cultured at 37° C. with 5% CO₂ for three weeks in 500 microliters of chondrogenic medium containing 500 nanograms per milliliter BMP-6 (R&D Systems, Minneapolis, Minn.) in addition to high glucose (25 millimolar), DMEM supplemented with 10 nanograms per milliliter TGF-beta-3, 10⁻⁷ molar Dexamethasone, 50 micrograms per milliliter ascorbate-2-phosphate, 40 micrograms per milliliter proline, 100 micrograms per milliliter pyruvate, and 50 milligrams per milliliter ITS+®Premix (Becton Dickinson, Lincoln Park, N.J.); 6.25 micrograms per milliliter insulin, 6.25 micrograms per milliliter transferrin, 6.25 nanograms per milliliter selenious acid, 1.25 milligrams per milliliter BSA, and 5.35 milligrams per milliliter linoleic acid).

Thereafter, the pellet was cultured in hyperchondrogenic medium containing high-glucose DMEM supplemented with 20 nanomolar beta-glycerol phosphate, 50 nanograms per milliliter thyroxine, 1 nanomolar Dexamethasone, 50 micrograms per milliliter ascorbate-2-phosphate, 40 micrograms per milliliter proline, 100 micrograms per milliliter pyruvate, and 50 milligrams per milliliter ITS+™Premix. The medium was replaced every 3 to 4 days for 6 weeks. For microscopy, the pellets were embedded in paraffin, cut into 5 micrometer sections. The sections were stained with Safranin-O (Richard Allan Scientific, Kalamazoo, Mich.) and Toluidine Blue (Richard Allan Scientific, Kalamazoo, Mich.) in sodium borate (Sigma, St. Louis, Mo.).

Immunostaining

Paraffin-embedded sections were deparaffinized using xylene and rehydrated through graded alcohols. The pellet was pre-treated with 25 milligrams per milliliter hyaluronidase (Sigma, St. Louis, Mo.) in PBS for 30 minutes at 37° C. for optimal antigen retrieval. Residual enzymatic activity was removed by washing in PBS and non-specific staining was blocked with PBS containing 10% normal goat serum for 1 hour at 25° C. Rabbit antibody against type II collagen, type X collagen, Integrin-binding bone sialoprotein (IBSP), or osteocalcin (Cosmo Bio, Japan; 1:500, 1:100, 1:100, 1:100 dilution respectively with PBS containing 1% BSA) was placed on the sections for 72 hours at 4° C. After extensive washing with PBS, the sections were incubated in biotinylated goat anti-rabbit (Vector Laboratories, Burlingame, Calif.; 1:500) for 1 hour at 25° C., washed and then incubated with streptavidin conjugated Texas Red (1:400; Vector Laboratories, Burlingame, Calif.) for 1 hour at 25° C. The slides were washed in PBS, dried, and coverslipped with anti-fade mounting medium containing DAPI (Vectashield, Vector Labs, Burlingame, Calif.). Immunostaining was visualized and photographed under Epifluorescence illumination with an E800 (Nikon, Japan) and SPOT RT camera and software (Diagnostic Instruments, Ml) or a DMRXA2 (Leica Microsystems, Pa.) equipped with a SensiCam CCD camera and Slidebook deconvolution software (Intelligent Imaging Innovations, Colo.).

RNA Isolation

Total RNA was prepared from 2 million undifferentiated MSCs at day 0, from 30 pellets each at 1, 2, and 3 weeks, and from 60 pellets each at 4, 5, and 6 weeks. Pellets incubated 7 days or longer were digested with 3 milligrams per milliliter collagenase, 1 milligram per milliliter hyaluronidase and 0.25% trypsin for about 3 hours at 37° C. Total RNA was extracted by using RNAqueous Kit (Ambion, Austin, Tex.).

RT-PCR

RNA was converted to cDNA and amplified by the Titan One Tube RT-PCR System (Roche Molecular Biochemicals, Germany). RT was performed by a 30 minute incubation at 50° C., followed by 2 minutes at 94° C. to inactivate the reverse transcriptase. PCR amplification conditions for the resulting cDNAs were performed by 35 cycles of 94° C. for 30 seconds, 58° C. for 45 seconds, and 68° C. for 45 seconds, in which the 68° C. step was increased by 5 seconds every cycle after 10 cycles. The reaction products were resolved by electrophoresis on a 1% agarose gel and visualized with ethidium bromide. PCR primers were as follows:

| | | |
|---|---|---|
| RUNX2/CBFA1 | 5′-AGGCAGTTCCCAAGCATTTC-3′ | |
| (forward) | SEQ ID NO:1 | |
| RUNX2/CBFA1 | 5′-GGTCGCCAAACAGATTCATC-3′ | 440 bp |
| (reverse) | SEQ ID NO:2 | |
| Osterix | 5′-TGCAGCAAATTTGGTGGCTC-3′ | |
| (forward) | SEQ ID NO:3 | |
| Osterix | 5′-AGCAAAGTCAGATGGGTAGG-3′ | 540 bp |
| (reverse) | SEQ ID NO:4 | |
| IBSP(forward) | 5′-CAGTAGTGACTCATCCGAAG-3′ | |
| | SEQ ID NO:5 | |
| IBSP(reverse) | 5′-GGAGAGGTTGTTGTCTTCGA-3′ | 507 bp |
| | SEQ ID NO:6 | |
| Osteocalcin | 5′-ACCGAGACACCATGAGAGCC-3′ | |
| (forward) | SEQ ID NO:7 | |
| Osteocalcin | 5′-GAAGAGGAAAGAAGGGTGCC-3′ | 383 bp |
| (reverse) | SEQ ID NO:8 | |
| beta-ACTIN | 5′-CCAAGGCCAACCGCGAGAAGATGAC-3′ | |
| (forward) | SEQ ID NO:9 | |
| beta-ACTIN | 5′-AGGGTACATGGTGGTGCCGCCAGAC-3′ | 587 bp |
| (reverse) | SEQ ID NO:10 | |

Microarray

Experimental procedures for microarray assays were performed according to the Affymetrix GeneChip Expression Analysis Technical Manual (Affymetrix, Santa Clara, Calif.). In brief, 5 micrograms of total RNA was used to synthesize double-stranded DNA (Superscript Choice System/Gibco BRL Life Technologies, Rockville, Md.). The DNA was purified using phenol/chloroform extraction with Phase Lock Gel (Eppendorf Scientific, N.Y.) and concentrated by ethanol precipitation. In vitro transcription was performed to produce biotin-labeled cRNA using a BioArray HighYield RNA Transcription Labeling Kit (Enzo Diagnostics, N.Y.) according to the manufacturer's instructions. Biotinylated cRNA was cleaned with an RNeasy Mini Kit (Qiagen, Valencia, Calif.), fragmented to 50 to 200 nucleotides, and hybridized 16 hours at 45° C. to Affymetrix HG-U95Av2 array, which contains approximately 12,000 human genes. After washing, the array was stained with streptavidin-phycoerythrin (Molecular Probes, Eugene, OR). The staining signal was amplified by biotinylated anti-streptavidin (Vector Laboratories, Burlingame, Calif.), followed by streptavidin-phycoerythrin, and then scanned on an HP GeneArray Scanner. The expression data was analyzed using Affymetrix MicroArray Suite v5.0 and Affymetrix Data Mining Tool v3.0. Signal intensities of all probe sets were scaled to the target value of 2500. All experiments were done in duplicate, and average signal intensities (SI) of the experiment pairs were used in fold change calculations.

Gene expression levels were measured at 0 days, 1 , 2, 3, 4, 5, and 6 weeks and fold changes (FCs) calculated from the levels in undifferentiated MSCs on day 0. To eliminate extremely high FC values, the value for SI was replaced by the corrected noise level (277) in FC calculations in instances in which one of the SI values was smaller that the highest noise level of the two subject arrays multiplied by a correction factor of 2.8.

The Results of the experiments presented in this Example are now described.

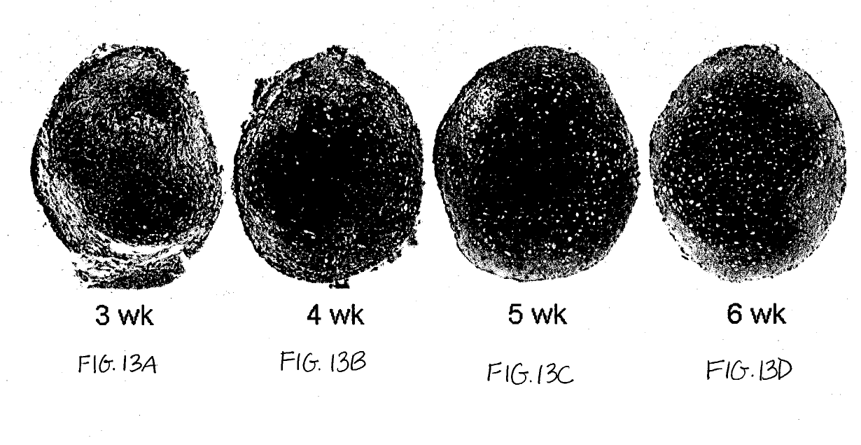

Differentiation into Hypertrophic Cells MSCs were cultured as micromass for 3 weeks in chondrogenic medium which contained TGF-beta 3, BMP-6, and 100 nanomolar Dexamethasone. As expected, the cells formed a cartilage pellet that contained proteoglycans (FIG. 13) and other cartilage components. After 3 weeks of initial culture as a micromass, the chondrogenic medium was replaced by hyperchondrogenic medium which contained beta-glycerol phosphate, thyroxine, and 1 nanomolar Dexamethasone. In preliminary experiments, only a few hypertrophic chondrocytes were obtained and the pellet cracked when the medium also contained 20 millimolar concentration of beta-glycerol phosphate employed by Mackay et al, Tissue Eng., 4(4):415-428 (1998). When the concentration of beta-glycerol phosphate was reduced one million fold to 20 nanomolar, hypertrophic chondrocytes began to appear after 4 weeks and increased in number for up to at least 6 weeks (FIG. 13). In contrast, when the pellets were continually cultured in chondrogenic medium for 6 weeks, the cells did not differentiate into hypertrophic chondrocytes. Also, supplementing the hyperchondrogenic medium with BMP-6 did not seem to have any effect.

Gene expression During Differentiation of MSCs

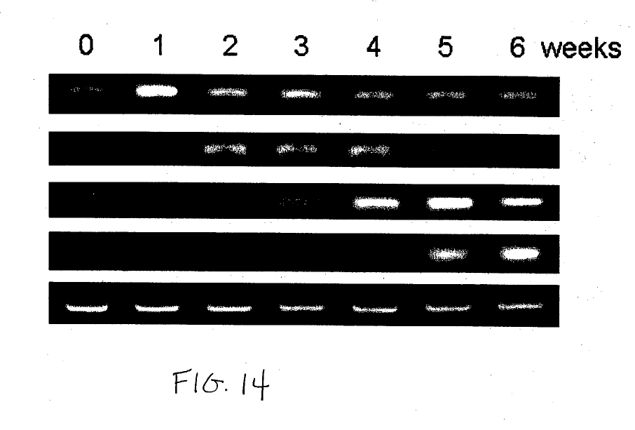

Assays by RT-PCR defined the time sequence of gene expression as the cells differentiated. mRNA for RUNX2/CBFA1, a transcription factor involved in the control of chondrocyte hypertrophy and osteoblastic differentiation, was present in undifferentiated MSCs and throughout the time course of differentiation (FIG. 14). Of special interest was that the levels for RUNX2/CBFA1 peaked before there was expression of the downstream transcription factor osterix that is essential for osteoblast differentiation. Osterix, in turn, reached a peak level before integrin binding bone sialoprotein (IBSP), an early marker for bone matrix, was expressed maximally. Also, IBSP reached a peak level before osteocalcin, a late marker for bone matrix, was expressed maximally.

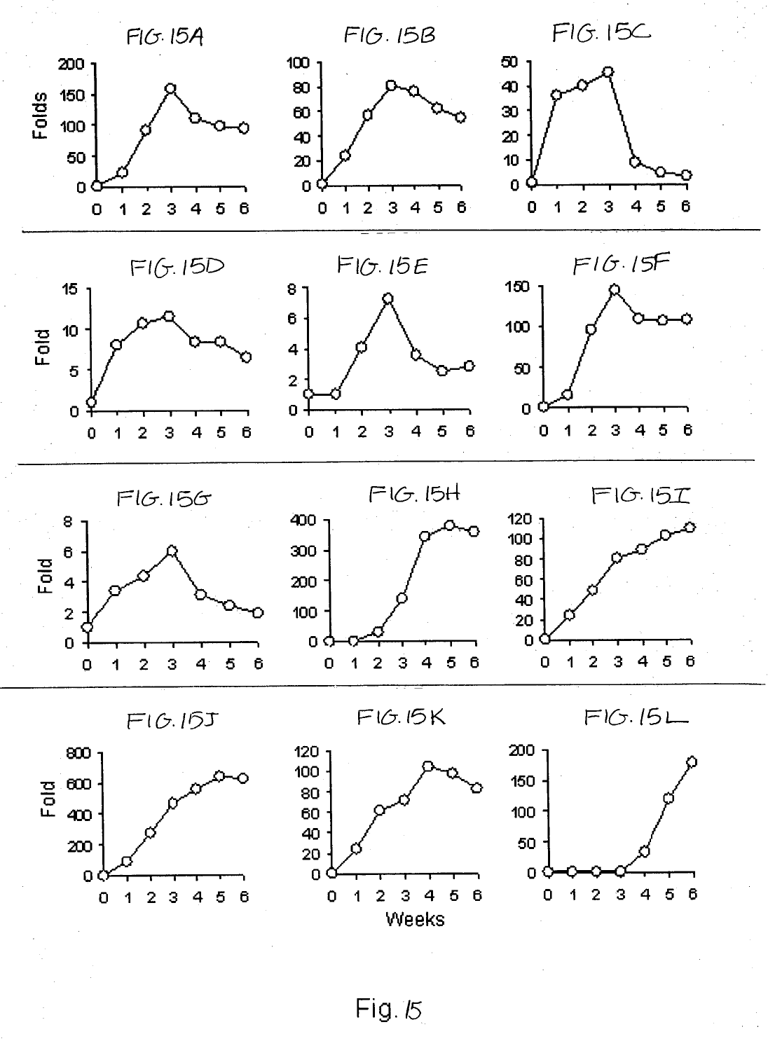

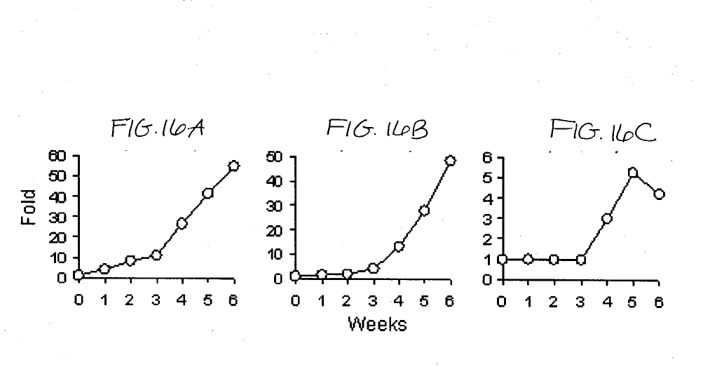

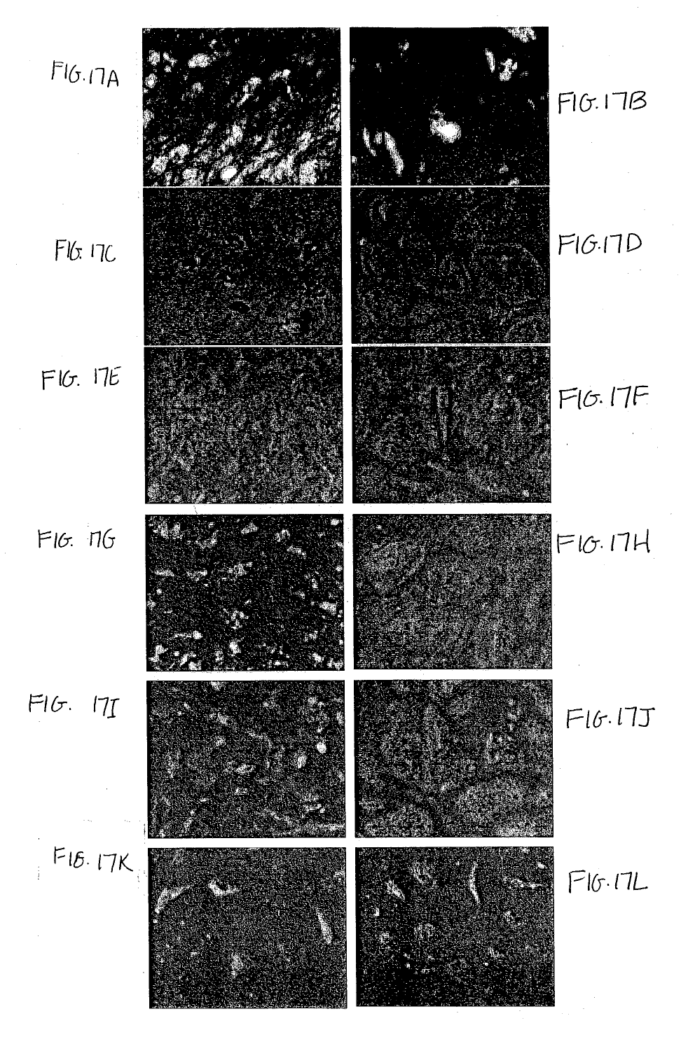

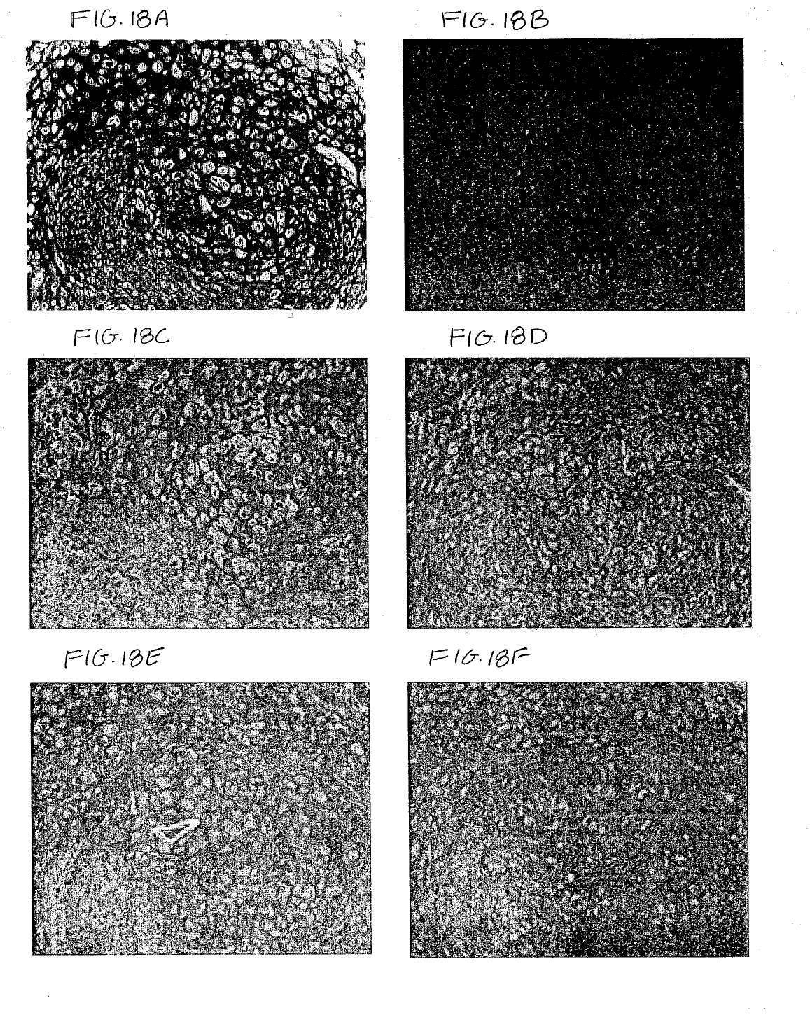

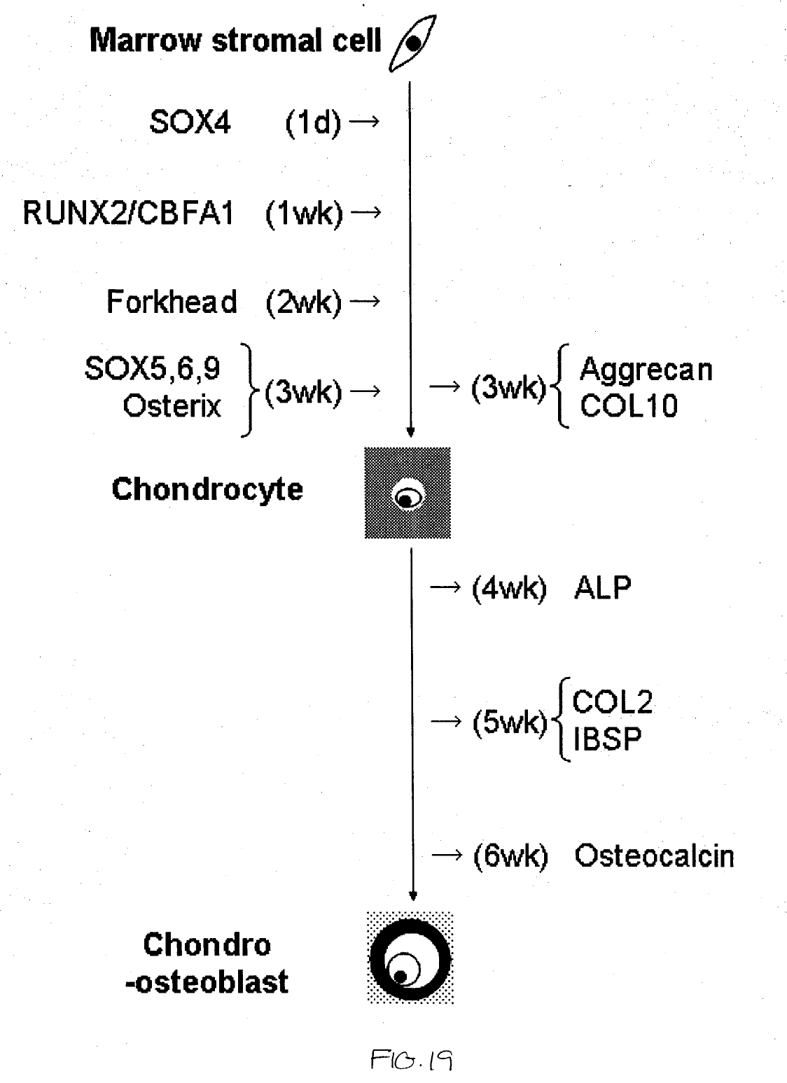

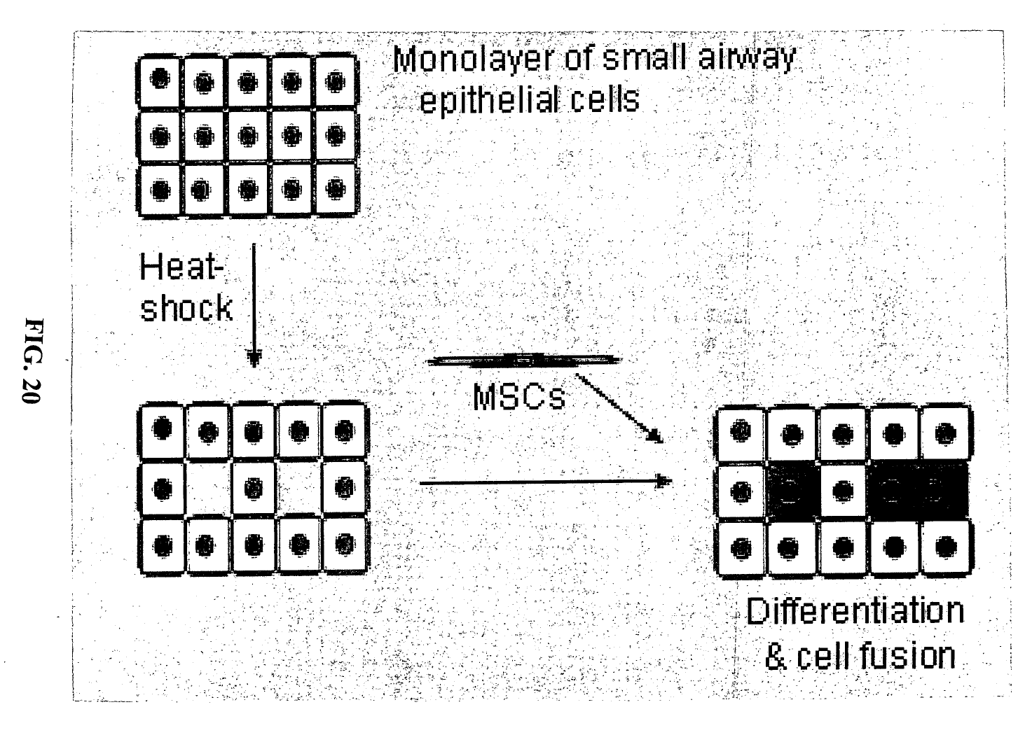

Data from microarray analyses were consistent with the RT-PCR data (FIG. 15). The time courses for the expression of most of chondro- and osteo-related mRNAs followed one of three general patterns; (a) mRNAs that reached peak levels within the first 3 weeks of incubation period in chondrogenesis medium; (b) mRNAs that continued to increase throughout the 3 weeks in chondrogenic medium and then for one or more weeks after the transfer to hyperchondrogenic medium; (c) mRNAs that were not expressed at impressive levels until the samples were transferred to the hyperchondrogenic medium. The mRNAs that peaked at 3 weeks included COL10A1, osteopontin, COMP, aggrecan, dermatan sulfate proteoglycan-3 (DSPG3), matrilin-3, and prolyl 4-hydroxylase alpha (II) (P4Hα2). The mRNAs that continued to increase included IBSP, parathyroid hormone/parathyroid hormone-related peptide receptor (PTHrPR), COL2A1, and alkaline phosphatase (ALP). The mRNAs that began to be expressed at significant levels after the transfer to hyperchondrogenic medium included osteocalcin, and mRNAs for a series of degradative enzymes: cathepsin 0, cathepsin H, and MMP-8 (neutrophil collagenase) (FIG. 16). The increases in the degradative enzymes were consistent with the necessary degradation of cartilage matrix that accompanies the same differentiation in vivo.

Immunohistochemical Analysis of Hypertrophic Cells

Immunostaining after 3 weeks demonstrated that type II and type X collagen were present throughout all regions of the extracellular matrix (FIG. 17) except for a thin ring around the periphery of the pellet. After 6 weeks, type II and type X collagen were still detected but only in the pericellular domains of the chondrocytes. Interestingly, IBSP and osteocalcin were also in the pericellular domains. Furthermore, the pericellular domains of the same cells contained type II collagen, type X collagen, IBSP, and osteocalcin (FIG. 18). The results indicated that the cells acquired an osteoblast phenotype in addition to chondrocyte phenotype after 6 weeks.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

What is claimed is:

1. A method of differentiating an adult bone marrow stromal cell into a desired cell type, said method comprising co-culturing said marrow stromal cell with another cell having said desired cell type, wherein said marrow stromal cell acquires the phenotype of said another cell.

2. The method of claim 1, wherein said another cell is damaged prior to coculturing with said adult bone marrow stromal cell.

3. The method of claim 2, wherein said damage is caused by a subjecting said other cell to a treatment selected from the group consisting of heat shock treatment, x-ray treatment, osmotic pressure, electroporation, and treatment with a toxin.

4. The method of claim 1, wherein said desired cell type is selected from the group consisting of a neuronal cell, an epithelial cell, a chondrocyte cell, a myocyte cell, an adipocyte cell, a thyroid cell, an adrenal cell, an endothelial cell, a cardiomyocyte, a renal cell, a hepatocyte, and a beta cell.

5. The method of claim 1, wherein said desired cell type is a neuronal cell.

6. The method of claim 1, wherein said desired cell type is an epithelial cell.

7. The method of claim 1, wherein said desired cell type is a chondrocyte cell.

8. The method of claim 1, wherein said desired cell type is a myocyte cell.

9. The method of claim 1, wherein said desired cell type is an adipocyte cell.

10. The method of claim 1, wherein said desired cell type is a beta cell.

11. The method of claim 1, wherein said desired cell type is a cardiomyocyte.

12. The method of claim 1, wherein said desired cell type is an endothelial cell.

13. The method of claim 1, wherein said desired cell type is a hepatocyte.

14. The method of claim 1, wherein said desired cell type is a renal cell.

15. The marrow stromal cell produced by the method of claim 1.

16. A method for improving recovery of a mammal from a neuronal injury, said method comprising culturing bone marrow stromal cells in a neurigenic medium and implanting said marrow stromal cells so cultured into the site of neuronal injury at least seven days post injury.

17. The method of claim 11, wherein said neurigenic medium comprises beta-3-mercaptoethanol.

18. The method of claim 11, wherein said neurigenic medium comprises dimethylsulfoxide and butylated hydroxyanisole.

19. A method of enhancing production of hypertrophic chondrocyte cells, said method comprising co-culturing marrow stromal cells with chondrocytes in a hyperchondrogenic medium, said medium comprising from about 10 nanomolar to about 100 nanomolar beta glycerol phosphate.

20. The method of claim 19, wherein said beta glycerol phosphate is present at about 20 nanomolar.

21. A hyperchondrogenic medium comprising about 20 nanomolar glycerol phosphate.

22. A method of inducing a bone marrow stromal cell to produce a protein, said method comprising:
(a) transfecting the bone marrow stromal cell with a DNA construct comprising: a splice site; a promoter for the expression of said protein; a marker gene; an internal ribosomal site (IRES); an antibiotic resistance gene; and another splice site;
(b) co-culturing said transfected bone marrow stromal cell with another damaged cell type obtained from an animal source;
(c) isolating those transfected bone marrow stromal cells that express both said marker gene and said antibiotic resistance gene; and
(d) optionally excising said DNA construct from said bone marrow stromal cell by incubation with recombinant Cre protein.

23. The method of claim 22, wherein said protein is insulin.

24. The method of claim 22, wherein said cell of interest is a beta cell.

25. A method of inducing a bone marrow stromal cell to produce insulin, said method comprising:
(a) transfecting the bone marrow stromal cell with a DNA construct comprising: a lox site; a promoter for the expression of insulin; a green fluorescent protein gene; an internal ribosomal site (IRES); a neomycin resistance gene; and another lox site;
(b) co-culturing said transfected bone marrow stromal cell with a heat-shocked beta cell obtained from an animal source;
(c) isolating those transfected bone marrow stromal cells that express both said green fluorescent protein gene and said neomycin resistance gene; and
(d) optionally excising said DNA construct from said bone marrow stromal cell by incubation with recombinant Cre protein.


SEQUENCE LISTING (10 sequences)

SEQ ID NO: 1 - DNA, 20 bp, Artificial
  Forward primer for RUNX2/CBFA1
aggcagttcccaagcatttc

SEQ ID NO: 2 - DNA, 20 bp, Artificial
  Reverse primer for RUNX2/CBFA1
ggtcgccaaacagattcatc

SEQ ID NO: 3 - DNA, 20 bp, Artificial
  forward primer for osterix
tgcagcaaatttggtggctc

SEQ ID NO: 4 - DNA, 20 bp, Artificial
  reverse primer for osterix
agcaaagtcagatgggtagg

SEQ ID NO: 5 - DNA, 20 bp, Artificial
  forward primer for IBSP
cagtagtgactcatccgaag

SEQ ID NO: 6 - DNA, 20 bp, Artificial
  reverse primer for IBSP
ggagaggttgttgtcttcga

SEQ ID NO: 7 - DNA, 20 bp, Artificial
  forward primer for osteocalcin
accgagacaccatgagagcc

SEQ ID NO: 8 - DNA, 20 bp, Artificial
  reverse primer for osteocalcin
gaagaggaaagaagggtgcc

SEQ ID NO: 9 - DNA, 25 bp, Artificial
  forward primer for beta-actin
ccaaggccaaccgcgagaagatgac

SEQ ID NO: 10 - DNA, 25 bp, Artificial
  reverse primer for beta actin
agggtacatggtggtgccgccagac